(19) United States
(12) Patent Application Publication
(10) Pub. No.: US 2010/0197699 A1
(43) Pub. Date: Aug. 5, 2010

(54) I-OXA-3-Azaspiro (4.5) Decan-2-One And 1-OXA-3, 8-Diazaspiro (4.5) Decan-2-One Derivatives For The Treatment of Eating Disorders

(75) Inventors: Jonathan Bentley, Verona (IT); Matteo Biagetti, Verona (IT); Francesca Cardullo, Verona (IT); Thorsten Genski, Verona (IT); Silvia Rosalia Kopf, Verona (IT); Colin Philip Leslie, Verona (IT); Sergio Melotto, Verona (IT); Mirco Meniconi, Verona (IT); Giancarlo Merlo, Verona (IT); Domenica Antonia Pizzi, Verona (IT); Catia Seri, Verona (IT)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(73) Assignee: Glaxo Group Limited

(21) Appl. No.: 12/524,038

(22) PCT Filed: Jan. 30, 2008
(86) PCT No.: PCT/EP08/51110

§ 371 Date: Mar. 15, 2010

(30) Foreign Application Priority Data
Feb. 1, 2007 (GB) 0701962.3
Oct. 24, 2007 (GB) 0720880.4

(51) Int. Cl. A61K 31/423; C07D 498/10; C07D 413/12; C07D 413/14; C07D 417/14; A61K 31/444; A61K 31/501; A61K 31/497; A61K 31/506; A61P 3/04; A61P 25/24

(52) U.S. Cl. 514/252.05; 546/19; 548/216; 546/15; 544/230; 514/278; 514/376; 514/255.05; 514/275

(57) ABSTRACT

The present invention relates to novel compounds of formula (I), or a pharmaceutically acceptable salt or solvate thereof,

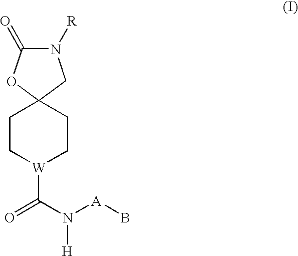

wherein
R is an aryl or heteroaryl; which may be substituted by one or more: halogen, C1-C4 alkyl, C1-C4 alkoxy, C1-C4 haloalkyl, C1-C4 haloalkoxy, cyano;
W is —CZ₁ or nitrogen;
Z₁ is hydrogen, C1-C4 alkyl;
A is a 5 membered heteroaryl, pyrazine, pyrimidine, or quinoline or quinazoline which may be substituted by one or more: halogen, C1-C4 alkyl, C1-C4 alkoxy, C1-C4 haloalkyl, C1-C4 haloalkoxy, cyano;
B is hydrogen or a 5-10 membered aryl or heteroaryl which may be substituted by one or more: halogen, C1-C4 alkyl, C1-C4 alkoxy, C1-C4 haloalkyl, C1-C4 haloalkoxy, cyano; being A and B linked via any atom;
   provided that when W is —CZ₁, compounds of formula (I) have a trans stereochemistry;
      processes for their preparation, intermediates used in these processes, pharmaceutical compositions containing them and their use in therapy, as NPY Y5 receptor antagonists and as agents for the treatment and/or prophylaxis of eating disorders such as a binge eating disorder.

The present invention relates to novel compounds, processes for their preparation, intermediates used in these processes, pharmaceutical compositions containing them and their use in therapy, as NPY Y5 receptor antagonists and as agents for the treatment and/or prophylaxis of eating disorders such as a binge eating disorder.

Neuropeptide Y (hereinafter referred to as NPY), a peptide consisting of 36 amino acids, was first isolated from porcine brain by Tatemoto et al. in 1982 [Nature, 296: 659 (1982)]. NPY is widely distributed in central and peripheral nervous systems and plays various roles as one of the most abundant peptides in the nervous system. NPY acts as an orexigenic substance in the central nervous system and markedly promotes fat accumulation via the mediation of the secretion of various hormones or the action of the nervous system. It is known that the continuous intracerebroventricular administration of NPY induces obesity and insulin resistance based on these actions (International Journal of Obesity, vol. 19: 517 (1995); Endocrinology, vol. 133: 1753 (1993)). It is also known that NPY has central effects that are related to diseases such as depression, anxiety, schizophrenia, pain, dementia and the like (Drugs, vol. 52, 371 (1996). Furthermore, in the periphery, NPY coexists with norepinephrine in sympathetic nerve endings and is involved in the tonicity of the sympathetic nervous system. It is known that peripheral administration of NPY causes vasoconstriction and enhances the activities of other vasoconstrictive substances such as norepinephrine (British Journal of Pharmacology, vol. 95: 419 (1988)). It is also reported that NPY could participate in the development of cardiac hypertrophy as a result of the sympathetic stimulation (Proceeding National Academic Science USA, Vol. 97, 1595 (2000)).

Endogenous receptor proteins that bind NPY and related peptides as ligands have been identified and distinguished, and several such proteins have been cloned and expressed. Six different receptor subtypes [Y1, Y2, Y3, Y4(PP), Y5, Y6] are recognised today based upon binding profile, pharmacology and/or composition if identity is known.

The Y5 subtype was isolated, characterized and reported recently in U.S. Pat. No. 5,602,024 (WO 96/16542). The effects mediated by the NPY Y5 receptor include eating stimulation and accumulation of fat (Nature, vol. 382, 168 (1996)); American Journal of Physiology, vol. 277, R1428 (1999)). It is reported that the NPY Y5 receptor also mediates some CNS effects, such as seizure and epilepsy, or pain and morphine withdrawal symptoms (Natural Medicine, vol. 3, 761 (1997); Proceeding Academic Science USA, vol. 96, 13518 (1999); The Journal of Pharmacology and Experimental Therapetics, vol. 284, 633 (1998)). In the periphery, the Y5 receptor is reported to be involved in diuresis and the hypoglycemic effect caused by NPY (British Journal of Pharmacology, vol. 120, 1335 (1998); Endocrinology, vol. 139, 3018 (1998)). NPY is also reported to enhance cardiac hypertrophy as a result of sympathetic accentuation (Proceeding National Academic Science USA, Vol. 97, 1595 (2000)).

The effects of NPY occur by binding to the NPY receptors in the central or peripheral nervous system. Therefore, the action of NPY can be prevented by blocking the binding to NPY receptors. Substances that antagonize NPY binding to NPY receptors may be useful for the prophylaxis or treatment of various diseases related to NPY, such as cardiovascular disorders (for example hypertension, nephropathy, heart disease, vasospasm), central nervous system disorders (for example bulimia, binge eating, depression, anxiety, seizure, epilepsy, dementia, pain, alcoholism, drug withdrawal), metabolic diseases (for example obesity, diabetes, hormone abnormality), sexual and reproductive dysfunction, gastro-intestinal motility disorder, respiratory disorder, inflammation or glaucoma and the like (Trends in Pharmacological Sciences, 15: 153 (1994); Life Science, 55, 551 (1994); Drugs, vol. 52, 371 (1996); The Journal of Allergy and Immunology, vol. 101, S345 (1998); Nature, vol. 396, 366 (1998); The Journal of Pharmacology and Experimental Therapeutics, vol. 284, 633 (1998); Trends in Pharmacological Science, vol. 20, 104 (1999); Proceeding National Academic Science USA, vol. 97, 1595 (2000)).

The object of the present invention is to provide novel compounds of general formula (I) or a pharmaceutically acceptable salt or solvate thereof:

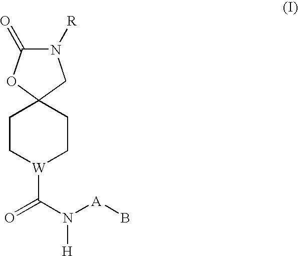

wherein
R is an aryl or heteroaryl; which may be substituted by one or more: halogen, C1-C4 alkyl, C1-C4 alkoxy, C1-C4 haloalkyl, C1-C4 haloalkoxy, cyano;
W is —CZ₁ or nitrogen;
Z₁ is hydrogen, C1-C4 alkyl;
A is a 5 membered heteroaryl, pyrazine, pyrimidine, or quinoline or quinazoline which may be substituted by one or more: halogen, C1-C4 alkyl, C1-C4 alkoxy, C1-C4 haloalkyl, C1-C4 haloalkoxy, cyano;
B is hydrogen or a 5-10 membered aryl or heteroaryl which may be substituted by one or more: halogen, C1-C4 alkyl, C1-C4 alkoxy, C1-C4 haloalkyl, C1-C4 haloalkoxy, cyano; being A and B linked via any atom;
   provided that when W is —CZ₁, compounds of formula (I) have a trans stereochemistry.

In one embodiment of the present invention, compounds of formula (Ia) are provided, which correspond to compounds of formula (I) in which W is —CZ₁ and in which the stereochemistry is trans. Trans stereochemistry is due to highest priority groups, according to Kahn-Prelog-Ingold classification, attached to the cyclohexane ring being on opposite sides of the cyclohexane ring. Trans stereochemistry can be designated also as “trans configuration” or “anti”; in the case of formula (I)′ the description (5r,8r) can also be used to describe the trans stereochemistry.

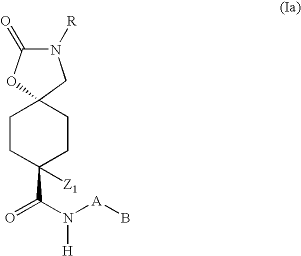

In one embodiment, R is a phenyl group. In another embodiment, R is a pyridine group. In a further embodiment R is a pyridazine group.

In one embodiment, Z₁ is hydrogen.

In one embodiment, A is a 5-membered heteroaryl selected among furyl, thiophenyl, pyrrolyl, oxazolyl, isooxazolyl, pyrazolyl, imidazolyl, thiazolyl, isothiazolyl, triazolyl.

In one embodiment, B is a 5-10 heteroaryl selected among furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, triazolyl, tetrazolyl, quinazolinyl, and benzodioxolyl. In another embodiment B is a 5-6 membered heteroaryl selected among furyl, thiophenyl, pyrrolyl, pyridyl, oxazolyl, isooxazolyl, pyrazolyl, imidazolyl, thiazolyl, isothiazolyl, pyridazinyl, pyrimidinyl, pyrazinyl, triazinyl, triazolyl, tetrazolyl.

In one embodiment, B is a phenyl.

The compounds of the present invention may be in the form of and/or may be administered as a pharmaceutically acceptable salt. For a review on suitable salts see Berge et al, J. Pharm. Sci., 1977, 66, 1-19.

Typically, a pharmaceutically acceptable salt may be readily prepared by using a desired acid or base as appropriate. The salt may precipitate from solution and be collected by filtration or may be recovered by evaporation of the solvent.

Suitable pharmaceutically acceptable addition salts are formed from acids which form non-toxic salts and examples are hydrochloride, hydrobromide, hydroiodide, sulphate, bisulphate, nitrate, phosphate, hydrogen phosphate, acetate, maleate, malate, fumarate, lactate, tartrate, citrate, formate, gluconate, succinate, pyruvate, oxalate, oxaloacetate, trifluoroacetate, saccharate, benzoate, methanesulphonate, ethanesulphonate, benzenesulphonate, p-toluenesulphonate and isethionate.

Pharmaceutically acceptable base salts include ammonium salts, alkali metal salts such as those of sodium and potassium, alkaline earth metal salts such as those of calcium and magnesium and salts with organic bases, including salts of primary, secondary and tertiary amines, such as isopropylamine, diethylamine, ethanolamine, trimethylamine, dicyclohexyl amine and N-methyl-D-glucamine.

Pharmaceutical acceptable salts may also be prepared from other salts, including other pharmaceutically acceptable salts, of the compound of formula (I) using conventional methods.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates of the compound of the invention are within the scope of the invention.

In addition, prodrugs are also included within the context of this invention. As used herein, the term “prodrug” means a compound which is converted within the body, e.g. by hydrolysis in the blood, into its active form that has medical effects. Pharmaceutically acceptable prodrugs are described in T. Higuchi and V. Stella, Prodrugs as Novel Delivery Systems, Vol. 14 of the A.C.S. Symposium Series, Edward B. Roche, ed., Bioreversible Carriers in Drug Design, American Pharmaceutical Association and Pergamon Press, 1987, and in D. Fleisher, S. Ramon and H. Barbra “Improved oral drug delivery: solubility limitations overcome by the use of prodrugs”, Advanced Drug Delivery Reviews (1996) 19(2) 115-130, each of which are incorporated herein by reference.

The term prodrug also encompasses any covalently bonded carriers that release a compound of structure (I) in vivo when such a prodrug is administered to a patient. Prodrugs are generally prepared by modifying functional groups in a way such that the modification is cleaved, either by routine manipulation or in vivo, yielding the parent compound. Prodrugs include, for example, compounds of this invention wherein amine groups are bonded to any group that, when administered to a patient, cleaves to form the amine groups. Thus, representative examples of prodrugs include (but are not limited to) acetate, formate and benzoate derivatives of amine functional groups of the compounds of structure (I).

With regard to stereoisomers, the compounds of general formula (I) may have one or more asymmetric carbon atoms and may occur as racemates, racemic mixtures and as individual enantiomers or diastereomers. All such isomeric forms are included within the present invention, including mixtures thereof.

When a specific enantiomer of a compound of general formula (I) is required, this may be obtained for example by resolution of a corresponding enantiomeric mixture of a compound of formula (I) using conventional methods, such as H.P.L.C. of the corresponding racemate using a suitable chiral support or by fractional crystallisation of the diastereoisomeric salts formed by reaction of the corresponding racemate with a suitable optically active acid or base, as appropriate.

Or a specific enantiomer may also be prepared from a corresponding optically pure intermediate.

Separation of diastereoisomers or cis and trans isomers or syn and anti isomers may be achieved by conventional techniques, e.g. by fractional crystallisation, chromatography or H.P.L.C. of a stereoisomeric mixture.

Furthermore, some of the crystalline forms of the compounds of structure (I) may exist as polymorphs, which are included in the present invention.

The term C1-C4 alkyl as used herein as a group or a part of the group refers to a linear or branched alkyl group containing from 1 to 4 carbon atoms; examples of such groups include methyl, ethyl, propyl, isopropyl, n-butyl, isobutyl, tert butyl.

The term halogen refers to a fluorine, chlorine, bromine or iodine atom.

The term halo C1-C4 alkyl means an alkyl group having one to 4 carbon atoms and wherein at least one hydrogen atom is replaced with halogen such as for example a trifluoromethyl group and the like.

The term C1-C4 alkoxy group may be a linear or a branched chain alkoxy group, for example methoxy, ethoxy, propoxy, prop-2-oxy, butoxy, but-2-oxy or methylprop-2-oxy and the like.

The term halo C1-C4 alkoxy group may be a C1-C4 alkoxy group as defined before substituted with at least one halogen, preferably fluorine, such as OCHF₂, or OCF₃.

The term aryl means an aromatic carbocyclic moiety such as phenyl, biphenyl or naphthyl.

The term heteroaryl means an aromatic heterocycle ring of 5 to 10 members and having at least one heteroatom selected from nitrogen, oxygen and sulfur, and containing at least 1 carbon atom, including both mono- and bicyclic ring systems.

Representative heteroaryls include (but are not limited to) furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, triazolyl, tetrazolyl, quinazolinyl, and benzodioxolyl.

In one aspect, the present invention provides compounds of formula (II) their pharmaceutically acceptable salt or solvate thereof,

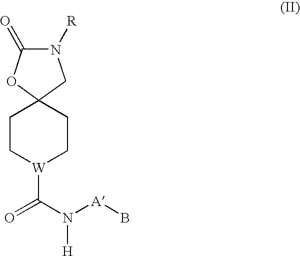

wherein
R is an aryl or heteroaryl; which may be substituted by one or more: halogen, C1-C4 alkyl, C1-C4 alkoxy, C1-C4 haloalkyl, C1-C4 haloalkoxy, cyano;
A′ is a thiazole which may be substituted by one or more: halogen, C1-C4 alkyl, C1-C4 alkoxy, C1-C4 haloalkyl, C1-C4 haloalkoxy, cyano;
W is —CZ₁ or nitrogen;

Z₁ is hydrogen, C1-C4 alkyl;
B is hydrogen or a 5-10 membered aryl or heteroaryl which may be substituted by one or more: halogen, C1-C4 alkyl, C1-C4 alkoxy, C1-C4 haloalkyl, C1-C4 haloalkoxy, cyano;
   provided that when W is —CZ₁, compounds of formula (I) have a trans stereochemistry.

In one embodiment W is —CZ₁ and Z₁ corresponds to hydrogen.

In another aspect, the present invention provides compounds of formula (IIa) their pharmaceutically acceptable salt or solvate thereof,

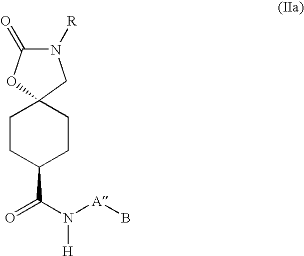

wherein
R is an aryl or heteroaryl; which may be substituted by one or more: halogen, C1-C4 alkyl, C1-C4 alkoxy, C1-C4 haloalkyl, C1-C4 haloalkoxy, cyano;
A″ is a pyrazole which may be substituted by one or more: halogen, C1-C4 alkyl, C1-C4 alkoxy, C1-C4 haloalkyl, C1-C4 haloalkoxy, cyano;
B is hydrogen or a 5-10 membered aryl or heteroaryl which may be substituted by one or more: halogen, C1-C4 alkyl, C1-C4 alkoxy, C1-C4 haloalkyl, C1-C4 haloalkoxy, cyano.

In a further aspect, the present invention provides compounds of formula (IIb) their pharmaceutically acceptable salt or solvate thereof,

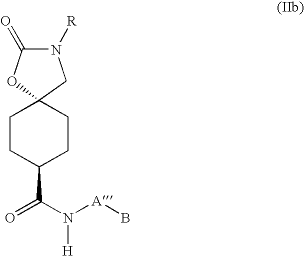

wherein
R is an aryl or heteroaryl; which may be substituted by one or more: halogen, C1-C4 alkyl, C1-C4 alkoxy, C1-C4 haloalkyl, C1-C4 haloalkoxy, cyano;
A′″ is a pyrazine which may be substituted by one or more: halogen, C1-C4 alkyl, C1-C4 alkoxy, C1-C4 haloalkyl, C1-C4 haloalkoxy, cyano;
B is hydrogen or a 5-10 membered aryl or heteroaryl which may be substituted by one or more: halogen, C1-C4 alkyl, C1-C4 alkoxy, C1-C4 haloalkyl, C1-C4 haloalkoxy, cyano.

In a further aspect, the present invention provides compounds of formula (IIc) their pharmaceutically acceptable salt or solvate thereof,

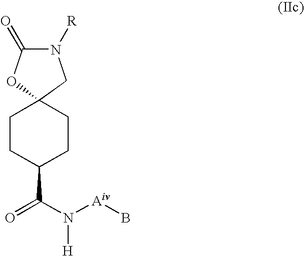

wherein
R is an aryl or heteroaryl; which may be substituted by one or more: halogen, C1-C4 alkyl, C1-C4 alkoxy, C1-C4 haloalkyl, C1-C4 haloalkoxy, cyano;
A^{iv} is a pyrimidine which may be substituted by one or more: halogen, C1-C4 alkyl, C1-C4 alkoxy, C1-C4 haloalkyl, C1-C4 haloalkoxy, cyano;
B is hydrogen or a 5-10 membered aryl or heteroaryl which may be substituted by one or more: halogen, C1-C4 alkyl, C1-C4 alkoxy, C1-C4 haloalkyl, C1-C4 haloalkoxy, cyano.

In a further aspect, the present invention provides compounds of formula (IId) their pharmaceutically acceptable salt or solvate thereof.

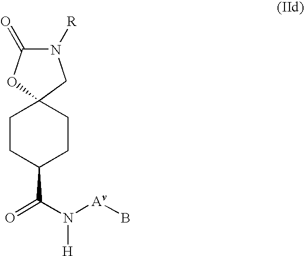

wherein

R is an aryl or heteroaryl; which may be substituted by one or more: halogen, C1-C4 alkyl, C1-C4 alkoxy, C1-C4 haloalkyl, C1-C4 haloalkoxy, cyano;

A^{v} is a quinoline which may be substituted by one or more: halogen, C1-C4 alkyl, C1-C4 alkoxy, C1-C4 haloalkyl, C1-C4 haloalkoxy, cyano;

B is hydrogen or a 5-10 membered aryl or heteroaryl which may be substituted by one or more: halogen, C1-C4 alkyl, C1-C4 alkoxy, C1-C4 haloalkyl, C1-C4 haloalkoxy, cyano.

In a further aspect, the present invention provides compounds of formula (IIe) their pharmaceutically acceptable salt or solvate thereof,

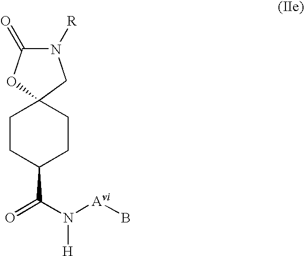

wherein
R is an aryl or heteroaryl; which may be substituted by one or more: halogen, C1-C4 alkyl, C1-C4 alkoxy, C1-C4 haloalkyl, C1-C4 haloalkoxy, cyano;
A^{vi} is a quinazoline which may be substituted by one or more: halogen, C1-C4 alkyl, C1-C4 alkoxy, C1-C4 haloalkyl, C1-C4 haloalkoxy, cyano;
B is hydrogen or a 5-10 membered aryl or heteroaryl which may be substituted by one or more: halogen, C1-C4 alkyl, C1-C4 alkoxy, C1-C4 haloalkyl, C1-C4 haloalkoxy, cyano.

### Example compounds of the present invention include

2-oxo-3-phenyl-N-[4-(2-pyridinyl)-1,3-thiazol-2-yl]-1-oxa-3,8-diazaspiro[4.5]decane-8-carboxamide;
3-(3,4-dichlorophenyl)-2-oxo-N-[4-(2-pyridinyl)-1,3-thiazol-2-yl]-1-oxa-3,8-diazaspiro-[4.5]decane-8-carboxamide;
(trans)-2-oxo-3-phenyl-N-[4-(2-pyridinyl)-1,3-thiazol-2-yl]-1-oxa-3-azaspiro[4.5]decane-8-carboxamide;
(trans)-2-oxo-3-(2-pyridinyl)-N-[4-(2-pyridinyl)-1,3-thiazol-2-yl]-1-oxa-3-azaspiro[4.5]-decane-8-carboxamide;
(trans)-N-[1-(2-fluorophenyl)-1H-pyrazol-3-yl]-2-oxo-3-phenyl-1-oxa-3-azaspiro[4.5]-decane-8-carboxamide;
(trans)-N-[1-(2-fluorophenyl)-1H-pyrazol-3-yl]-2-oxo-3-(3-pyridinyl)-1-oxa-3-azaspiro[4.5]-decane-8-carboxamide;
(trans)-N-[1-(2-fluorophenyl)-1H-pyrazol-3-yl]-2-oxo-3-(3-pyridazinyl)-1-oxa-3-azaspiro-[4.5]decane-8-carboxamide;
(trans)-2-oxo-3-phenyl-N-(5-phenyl-2-pyrazinyl)-1-oxa-3-azaspiro[4.5]decane-8-carboxamide;
(trans)-N-(5-bromo-2-pyrazinyl)-2-oxo-3-phenyl-1-oxa-3-azaspiro[4.5]decane-8-carboxamide;
(trans)-2-oxo-3-phenyl-N-[5-(1,3-thiazol-2-yl)-2-pyrazinyl]-1-oxa-3-azaspiro[4.5]decane-8-carboxamide;
(trans)-N-[5-(2-fluorophenyl)-2-pyrazinyl]-2-oxo-3-phenyl-1-oxa-3-azaspiro[4.5]decane-8-carboxamide;
(trans)-2-oxo-3-phenyl-N-[5-(2-pyridinyl)-2-pyrazinyl]-1-oxa-3-azaspiro[4.5]decane-8-carboxamide;
(trans)-N-[5-(2-Fluorophenyl)-2-pyrimidinyl]-2-oxo-3-phenyl-1-oxa-3-azaspiro[4.5]decane-8-carboxamide;
(trans)-2-oxo-3-phenyl-N-3-quinolinyl-1-oxa-3-azaspiro[4.5]decane-8-carboxamide;
(trans)-2-oxo-3-(2-pyridinyl)-N-3-quinolinyl-1-oxa-3-azaspiro[4.5]decane-8-carboxamide;
(trans)-2-oxo-3-phenyl-N-2-quinoxalinyl-1-oxa-3-azaspiro[4.5]decane-8-carboxamide;
   or pharmaceutically acceptable salts thereof.

In general, the compounds of structure (I) may be made according to the organic synthesis techniques known to those skilled in this field, as well as by the representative methods set forth in the Examples.

Compounds of formula (I), and salts and solvates thereof, may be prepared by the general methods outlined hereinafter. In the following description, the groups R, Z₁, W, A and B have the meanings as previously defined for compounds of formula (I) unless otherwise stated.

Compounds of formula (I) may be conveniently prepared, starting from compounds of formula (II), according to the following Scheme 1:

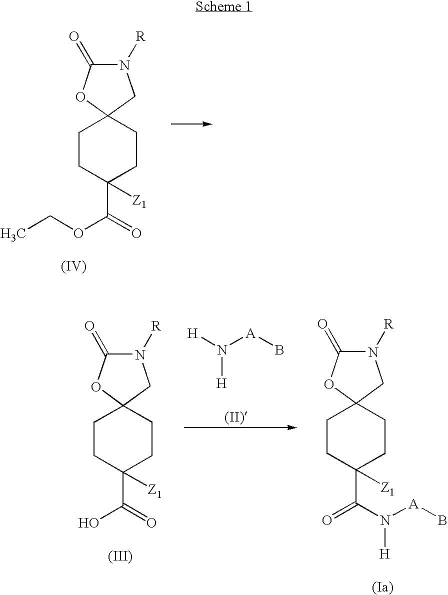

Compounds of formula (Ia) can be prepared by coupling of amines of formula (II)′ and carboxylic acids of formula (III). Standard conditions for such a transformation are described in Tetrahedron, 2005, 61(46), 10827-10852 and the references therein. Carboxylic acids of formula (III) can be prepared from esters of formula (IV) via hydrolysis with a reagent such as lithium hydroxide or sodium hydroxide in a suitable solvent (e.g. methanol/water or THF/water) followed by acidification with an acid (e.g. hydrochloric acid).

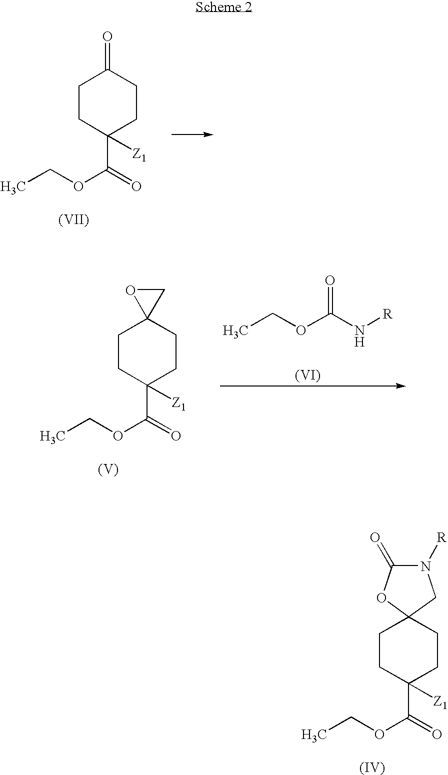

Esters of formula (IV) can be prepared from an epoxide of formula (V) and a carbamate of formula (VI) in a solvent such as HPMA, DMPU or NMP in the presence of a base such as sodium tertiary-butoxide, sodium hydride or BEMP, preferably at a temperature greater than 100° C. An epoxide of formula (VI) can be prepared from a ketone (VII), which is commercially available from e.g. Sigma-Aldrich Chemicals; carbamates of formula (VI) are commercially available from e.g. Sigma-Aldrich Chemicals.

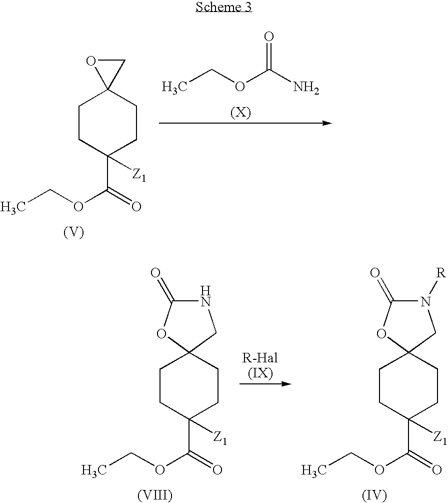

Esters of formula (IV) can be prepared from esters of formula (VIII) and an aryl halide of formula (IX). Suitable reactions conditions have been described in ‘Metal-Catalyzed Cross-Coupling Reactions (2nd Edition)’, 2004, 2, 699-760; Angewandte Chemie, International Edition, 2003, 42(44), 5400-5449 and the references therein. Aryl halides of formula (IX) are commercially available from e.g. Sigma-Aldrich Chemicals. Esters of formula (VIII) can be prepared from an epoxide of formula (V) and a carbamate of formula (X) in a solvent such as HPMA, DMPU or NMP in the presence of a base such as sodium tertiary-butoxide, sodium hydride or BEMP, preferably at a temperature greater than 100° C. A carbamate of formula (X) is commercially available from e.g. Sigma-Aldrich Chemicals.

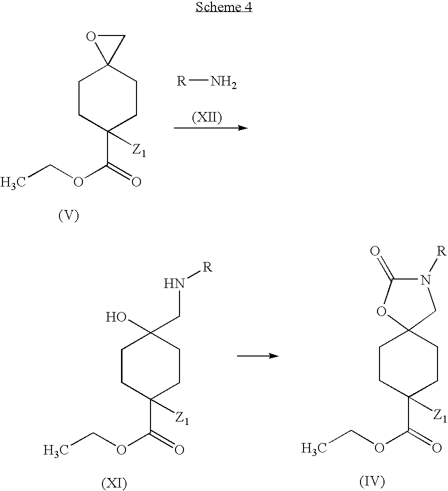

Alternatively, esters of formula (IV) can be prepared from amino-alcohols of formula (XI) and a reagent such as phosgene, triphosgene, carbonyl di-imidazole, disuccinimidyl carbonate, carbon dioxide, an alkylchloroformate e.g. benzyl chloroformate or ethyl chloroformate, an aryl chloroformate e.g. phenyl chloroformate or a dialkyl pyrocarbonate e.g. di-tertiary-butyl di-carbonate (Boc anhydride), optionally in the presence of a base such as triethylamine in a solvent such as dichloromethane. Amino-alcohols of formula (XI) can be prepared from an epoxide of formula (V) and amines of formula (XII) in a protic solvent such as tertiary-butanol or ethoxyethanol at temperatures greater than 100° C. Amines of formula (XII), such as aniline, are commercially available from e.g. Sigma-Aldrich Chemicals.

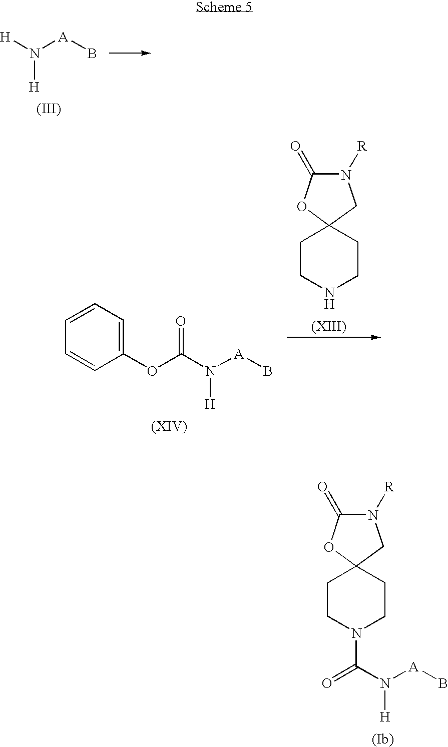

Compounds of formula (Ib), corresponding to compounds of formula (I) where W═N, can be prepared from amines of formula (XIII) and carbamates of formula (XIV) in the presence of a base, such as di-isopropylethylamine or triethylamine, in a solvent, such as acetonitrile, optionally with heating to 60° C. The preparation of amines of formula (XIII) is described in the Journal of Medicinal Chemistry, 1995, 38(19), 3772-9, U.S. Pat. No. 4,244,961 and WO9711940. Carbamates of formula (XIV) can be prepared from amines of formula (III) and a reagent such as phenyl chloroformate according to conditions described in Tetrahedron, 2005, 61(46), 10827-10852 and the references therein.

Those skilled in the art will appreciate that in the preparation of the compound of the invention or a solvate thereof it may be necessary and/or desirable to protect one or more sensitive groups in the molecule to prevent undesirable side reactions. Suitable protecting groups for use according to the present invention are well known to those skilled in the art and may be used in a conventional manner. See, for example, “Protective groups in organic synthesis” by T. W. Greene and P. G. M. Wuts (John Wiley & sons 1991) or “Protecting Groups” by P. J. Kocienski (Georg Thieme Verlag 1994). Examples of suitable amino protecting groups include acyl type protecting groups (e.g. formyl, trifluoroacetyl, acetyl), aromatic urethane type protecting groups (e.g. benzyloxycarbonyl (Cbz) and substituted Cbz), aliphatic urethane protecting groups (e.g. 9-fluorenylmethoxycarbonyl (Fmoc), t-butyloxycarbonyl (Boc), isopropyloxycarbonyl, cyclohexyloxycarbonyl) and alkyl type protecting groups (e.g. benzyl, trityl, chlorotrityl). Examples of suitable oxygen protecting groups may include for example trialkylsilyl groups, such as trimethylsilyl or tert-butyldimethylsilyl; alkyl ethers such as tetrahydropyranyl or tert-butyl; or esters such as acetate.

The subject invention also includes isotopically-labelled compounds, which are identical to those recited in formula (I) and following, but for the fact that one or more atoms are replaced by an atom having an atomic mass or mass number different from the atomic mass or mass number usually found in nature. Examples of isotopes that can be incorporated into compounds of the invention and pharmaceutically acceptable salts thereof include isotopes of hydrogen, carbon, nitrogen, oxygen, phosphorous, sulphur, fluorine, iodine, and chlorine, such as ²H, ³H, ¹¹C, ¹³C, ¹⁴C, ¹⁵N, ¹⁷O, ¹⁸O, ³¹P, ³²P, ³⁵S, ¹⁸F, ³⁶Cl, ¹²³I and ¹²⁵I.

Compounds of the present invention and pharmaceutically acceptable salts of said compounds that contain the aforementioned isotopes and/or other isotopes of other atoms are within the scope of the present invention. Isotopically-labelled compounds of the present invention, for example those into which radioactive isotopes such as ³H, ¹⁴C are incorporated, are useful in drug and/or substrate tissue distribution assays. Tritiated, i.e., ³H, and carbon-14, i.e., ¹⁴C, u isotopes are particularly preferred for their ease of preparation and detectability. ¹¹C and ¹⁸F isotopes are particularly useful in PET (positron emission tomography), and ¹²⁵I isotopes are particularly useful in SPECT (single photon emission computerized tomography), all useful in brain imaging. Isotopically labelled compounds of formula I and following of this invention can generally be prepared by carrying out the procedures disclosed in the Schemes and/or in the Examples below, by substituting a readily available isotopically labelled reagent for a non-isotopically labelled reagent.

Compounds of the present invention are antagonists of the NPY Y5 receptor and as such are useful for the prevention and treatment of disorders or diseases associated with the Y5 receptor sub-type, in particular for the treatment of eating disorders such as obesity, anorexia nervosa and bulimia nervosa, and other abnormal conditions, such as diabetes, hypertension, hyperlipemia, hypercholesterolemia, congestive heart failure, renal dysfunction, sexual/reproductive disorders, depression, anxiety, shock, epileptic seizure, memory loss, sleep disturbance, pain, migraine, cerebral hemorrhage, nasal congestion, gastrointestinal disorders, arthritis and immunodeficiency syndrome.

Compounds of the present invention may also be used in combination with other anti-obesity agents for increased efficacy in the prevention and treatment of obesity. Such agents would include, but not be limited to: sibutramine; dexfenfluramine; leptin; growth hormone secretagogue antagonists such as those disclosed and specifically described in U.S. Pat. No. 5,536,716; melanocortin agonists such as elanotan II; Beta-3 agonists such as those disclosed and specifically described in patent publications WO94/18161, WO95/29159, WO97/46556, WO98/04526 and WO98/32753; 5HT-2 agonists; orexin antagonists; melanin concentrating hormone antagonists; galanin antagonists; CCK agonists; GLP-1 agonists; corticotrophin releasing hormone agonists; Y1 antagonists, and CB1 antagonists

More particularly, compounds of the present invention may be useful as agents for the treatment and/or prophylaxis of eating disorders such as a binge eating disorder.

The method of treatment of this invention comprises a method of antagonizing the Y5 receptor and treating Y5 receptor mediated diseases by administering to a patient in need of such treatment a non-toxic therapeutically effective amount of a compound of this invention that selectively antagonizes the Y5 receptor in preference to the other NPY receptors.

Within the context of the present invention, the terms describing some indications used herein are classified in the Diagnostic and Statistical Manual of Mental Disorders, 4th Edition, published by the American Psychiatric Association (DSM-IV) and/or the International Classification of Diseases, 10th Edition (ICD-10). The various subtypes of the disorders mentioned herein are contemplated as part of the present invention. Numbers in brackets after the listed diseases below refer to the classification code in DSM-IV.

Depression and mood disorders including Major Depressive Episode, Manic Episode, Mixed Episode and Hypomanic Episode; Depressive Disorders including Major Depressive Disorder, Dysthymic Disorder (300.4), Depressive Disorder Not Otherwise Specified (311); Other Mood Disorders including Mood Disorder Due to a General Medical Condition (293.83) which includes the subtypes With Depressive Features, With Major Depressive-like Episode, With Manic Features and With Mixed Features), Substance-Induced Mood Disorder (including the subtypes With Depressive Features, With Manic Features and With Mixed Features) and Mood Disorder Not Otherwise Specified (296.90);

Anxiety disorders including Panic Attack; Panic Disorder including Panic Disorder without Agoraphobia (300.01) and Panic Disorder with Agoraphobia (300.21); Agoraphobia; Agoraphobia Without History of Panic Disorder (300.22), Specific Phobia (300.29, formerly Simple Phobia) including the subtypes Animal Type, Natural Environment Type, Blood-Injection-Injury Type, Situational Type and Other Type), Social Phobia (Social Anxiety Disorder, 300.23), Obsessive-Compulsive Disorder (300.3), Posttraumatic Stress Disorder (309.81), Acute Stress Disorder (308.3), Generalized Anxiety Disorder (300.02), Anxiety Disorder Due to a General Medical Condition (293.84), Substance-Induced Anxiety Disorder, Separation Anxiety Disorder (309.21), Adjustment Disorders with Anxiety (309.24) and Anxiety Disorder Not Otherwise Specified (300.00);

Substance-related disorders including Substance Use Disorders such as Substance Dependence, Substance Craving and Substance Abuse; Substance-Induced Disorders such as Substance Intoxication, Substance Withdrawal, Substance-Induced Delirium, Substance-Induced Persisting Dementia, Substance-Induced Persisting Amnestic Disorder, Substance-Induced Psychotic Disorder, Substance-Induced Mood Disorder, Substance-Induced Anxiety Disorder, Substance-Induced Sexual Dysfunction, Substance-Induced Sleep Disorder and Hallucinogen Persisting Perception Disorder (Flashbacks); Alcohol-Related Disorders such as Alcohol Dependence (303.90), Alcohol Abuse (305.00), Alcohol Intoxication (303.00), Alcohol Withdrawal (291.81), Alcohol Intoxication Delirium, Alcohol Withdrawal Delirium, Alcohol-Induced Persisting Dementia, Alcohol-Induced Persisting Amnestic Disorder, Alcohol-Induced Psychotic Disorder, Alcohol-Induced Mood Disorder, Alcohol-Induced Anxiety Disorder, Alcohol-Induced Sexual Dysfunction, Alcohol-Induced Sleep Disorder and Alcohol-Related Disorder Not Otherwise Specified (291.9); Amphetamine (or Amphetamine-Like)-Related Disorders such as Amphetamine Dependence (304.40), Amphetamine Abuse (305.70), Amphetamine Intoxication (292.89), Amphetamine Withdrawal (292.0), Amphetamine Intoxication Delirium, Amphetamine Induced Psychotic Disorder, Amphetamine-Induced Mood Disorder, Amphetamine-Induced Anxiety Disorder, Amphetamine-Induced Sexual Dysfunction, Amphetamine-Induced Sleep Disorder and Amphetamine-Related Disorder Not Otherwise Specified (292.9); Caffeine Related Disorders such as Caffeine Intoxication (305.90), Caffeine-Induced Anxiety Disorder, Caffeine-Induced Sleep Disorder and Caffeine-Related Disorder Not Otherwise Specified (292.9); Cannabis-Related Disorders such as Cannabis Dependence (304.30), Cannabis Abuse (305.20), Cannabis Intoxication (292.89), Cannabis Intoxication Delirium, Cannabis-Induced Psychotic Disorder, Cannabis-Induced Anxiety Disorder and Cannabis-Related Disorder Not Otherwise Specified (292.9); Cocaine-Related Disorders such as Cocaine Dependence (304.20), Cocaine Abuse (305.60), Cocaine Intoxication (292.89), Cocaine Withdrawal (292.0), Cocaine Intoxication Delirium, Cocaine-Induced Psychotic Disorder, Cocaine-Induced Mood Disorder, Cocaine-Induced Anxiety Disorder, Cocaine-Induced Sexual Dysfunction, Cocaine-Induced Sleep Disorder and Cocaine-Related Disorder Not Otherwise Specified (292.9); Hallucinogen-Related Disorders such as Hallucinogen Dependence (304.50), Hallucinogen Abuse (305.30), Hallucinogen Intoxication (292.89), Hallucinogen Persisting Perception Disorder (Flashbacks) (292.89), Hallucinogen Intoxication Delirium, Hallucinogen-Induced Psychotic Disorder, Hallucinogen-Induced Mood Disorder, Hallucinogen-Induced Anxiety Disorder and Hallucinogen-Related Disorder Not Otherwise Specified (292.9); Inhalant-Related Disorders such as Inhalant Dependence (304.60), Inhalant Abuse (305.90), Inhalant Intoxication (292.89), Inhalant Intoxication Delirium, Inhalant-Induced Persisting Dementia, Inhalant-Induced Psychotic Disorder, Inhalant-Induced Mood Disorder, Inhalant-Induced Anxiety Disorder and Inhalant-Related Disorder Not Otherwise Specified (292.9); Nicotine-Related Disorders such as Nicotine Dependence (305.1), Nicotine Withdrawal (292.0) and Nicotine-Related Disorder Not Otherwise Specified (292.9); Opioid-Related Disorders such as Opioid Dependence (304.00), Opioid Abuse (305.50), Opioid Intoxication (292.89), Opioid Withdrawal (292.0), Opioid Intoxication Delirium, Opioid-Induced Psychotic Disorder, Opioid-Induced Mood Disorder, Opioid-Induced Sexual Dysfunction, Opioid-Induced Sleep Disorder and Opioid-Related Disorder Not Otherwise Specified (292.9); Phencyclidine (or Phencyclidine-Like)-Related Disorders such as Phencyclidine Dependence (304.60), Phencyclidine Abuse (305.90), Phencyclidine Intoxication (292.89), Phencyclidine Intoxication Delirium, Phencyclidine-Induced Psychotic Disorder, Phencyclidine-Induced Mood Disorder, Phencyclidine-Induced Anxiety Disorder and Phencyclidine-Related Disorder Not Otherwise Specified (292.9); Sedative-, Hypnotic-, or Anxiolytic-Related Disorders such as Sedative, Hypnotic, or Anxiolytic Dependence (304.10), Sedative, Hypnotic, or Anxiolytic Abuse (305.40), Sedative, Hypnotic, or Anxiolytic Intoxication (292.89), Sedative, Hypnotic, or Anxiolytic Withdrawal (292.0), Sedative, Hypnotic, or Anxiolytic Intoxication Delirium, Sedative, Hypnotic, or Anxiolytic Withdrawal Delirium, Sedative-, Hypnotic-, or Anxiolytic-Persisting Dementia, Sedative-, Hypnotic-, or Anxiolytic-Persisting Amnestic Disorder, Sedative-, Hypnotic-, or Anxiolytic-Induced Psychotic Disorder, Sedative-, Hypnotic-, or Anxiolytic-Induced Mood Disorder, Sedative-, Hypnotic-, or Anxiolytic-Induced Anxiety Disorder Sedative-, Hypnotic-, or Anxiolytic-Induced Sexual Dysfunction, Sedative-, Hypnotic-, or Anxiolytic-Induced Sleep Disorder and Sedative-, Hypnotic-, or Anxiolytic-Related Disorder Not Otherwise Specified (292.9); Polysubstance-Related Disorder such as Polysubstance Dependence (304.80); and Other (or Unknown) Substance-Related Disorders such as Anabolic Steroids, Nitrate Inhalants and Nitrous Oxide;

Sleep disorders including primary sleep disorders such as Dyssomnias such as Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47); primary sleep disorders such as Parasomnias such as Nightmare Disorder (307.47), Sleep Terror Disorder (307.46), Sleepwalking Disorder (307.46) and Parasomnia Not Otherwise Specified (307.47); Sleep Disorders Related to Another Mental Disorder such as Insomnia Related to Another Mental Disorder (307.42) and Hypersomnia Related to Another Mental Disorder (307.44); Sleep Disorder Due to a General Medical Condition; and Substance-Induced Sleep Disorder including the subtypes Insomnia Type, Hypersomnia Type, Parasomnia Type and Mixed Type;

Eating disorders such as Anorexia Nervosa (307.1) including the subtypes Restricting Type and Binge-Eating/Purging Type; Bulimia Nervosa (307.51) including the subtypes Purging Type and Nonpurging Type; Obesity; Compulsive Eating Disorder; Binge Eating Disorder; and Eating Disorder Not Otherwise Specified (307.50);

Sexual dysfunctions including Sexual Desire Disorders such as Hypoactive Sexual Desire Disorder (302.71), and Sexual Aversion Disorder (302.79); sexual arousal disorders such as Female Sexual Arousal Disorder (302.72) and Male Erectile Disorder (302.72); orgasmic disorders such as Female Orgasmic Disorder (302.73), Male Orgasmic Disorder (302.74) and Premature Ejaculation (302.75); sexual pain disorder such as Dyspareunia (302.76) and Vaginismus (306.51); Sexual Dysfunction Not Otherwise Specified (302.70); paraphilias such as Exhibitionism (302.4), Fetishism (302.81), Frotteurism (302.89), Pedophilia (302.2), Sexual Masochism (302.83), Sexual Sadism (302.84), Transvestic Fetishism (302.3), Voyeurism (302.82) and Paraphilia Not Otherwise Specified (302.9); gender identity disorders such as Gender Identity Disorder in Children (302.6) and Gender Identity Disorder in Adolescents or Adults (302.85); and Sexual Disorder Not Otherwise Specified (302.9).

In a further embodiment the invention provides the use of a compound of formula (I), or a pharmaceutically acceptable salt or solvate thereof, in the preparation of a medicament for the treatment of a binge eating disorder.

In a further embodiment the present invention provides a method of treatment of a mammal suffering from a binge eating disorder, which comprises administering to said subject an effective amount of a compound of formula (I) or a pharmaceutically acceptable salt or solvate thereof.

In a further embodiment the invention provides the use of a compound of formula (I), or a pharmaceutically acceptable salt or solvate thereof, in the preparation of a medicament for the treatment of obesity.

In a further embodiment the present invention provides a method of treatment of a mammal suffering from obesity, which comprises administering to said subject an effective amount of a compound of formula (I) or a pharmaceutically acceptable salt or solvate thereof.

Compounds of the general formula (I) can be administered orally or parenterally and may be formulated in the form suitable for administration to provide an agent for treatment of various diseases related to NPY, which include, for example, cardiovascular disorders (for example hypertension, nephropathy, heart disease, vasospasm, arteriosclerosis), central nervous system disorders (for example bulimia, depression, anxiety, seizure, epilepsy, dementia, pain, alcoholism, drug withdrawal), metabolic diseases (for example obesity, diabetes, hormone abnormality, hypercholesterolemia, hyperlipidemia), sexual and reproductive dysfunction, gastro-intestinal motility disorder, respiratory disorder, inflammation or glaucoma and the like, preferably, bulimia, obesity, diabetes and the like.

While it is possible that, for use in therapy a therapeutically effective amount of a compound of formula (I), or a pharmaceutically acceptable salt or solvate thereof, may be administered as the raw chemical, it is possible to present the active ingredient as a pharmaceutical composition. Thus, in a further embodiment the invention provides a pharmaceutical composition comprising a compound of formula (I), or a pharmaceutically acceptable salt or solvate thereof, in admixture with one or more pharmaceutically acceptable carriers, diluents, or excipients. The carrier(s), diluent(s) or excipient(s) must be acceptable in the sense of being compatible with the other ingredients of the formulation and not deleterious to the recipient thereof. In a further embodiment the invention also provides a process for the preparation of a pharmaceutical composition including admixing a compound of (I), or a pharmaceutically acceptable salt or solvate thereof, with one or more pharmaceutically acceptable carriers, diluents or excipients.

Pharmaceutical compositions of the invention may be formulated for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Therefore, the pharmaceutical compositions of the invention may be formulated, for example, as tablets, capsules, powders, granules, lozenges, creams or liquid preparations, such as oral or sterile parenteral solutions or suspensions. Such pharmaceutical formulations may be prepared by any method known in the art of pharmacy, for example by bringing into association the active ingredient with the carrier(s) or excipient(s).

Tablets and capsules for oral administration may be in unit dose presentation form, and may contain conventional excipients such as binding agents, for example syrup, acacia, gelatine, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatine, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

The topical formulations of the present invention may be presented as, for instance, ointments, creams or lotions, eye ointments and eye or ear drops, impregnated dressings and aerosols, and may contain appropriate conventional additives such as preservatives, solvents to assist drug penetration and emollients in ointments and creams. The formulations may also contain compatible conventional carriers, such as cream or ointment bases and ethanol or oeyl alcohol for lotions. Such carriers may be present as from about 1% up to about 98% of the formulation. More usually they will form up to about 80% of the formulation.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

Pharmaceutical formulations adapted for rectal administration may be presented as suppositories or as enemas.

Pharmaceutical formulations adapted for nasal administration wherein the carrier is a solid may include a coarse powder having a particle size for example in the range 20 to 500 microns which is administered in the manner in which snuff is taken, i.e. by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered, dose pressurised aerosols, nebulizers or insufflators.

Pharmaceutical formulations adapted for vaginal administration may be presented as pessaries, tampons, creams, gels, pastes, foams or spray formulations.

It should be understood that in addition to the ingredients particularly mentioned above, the formulations may include other agents conventional in the art having regard to the type of formulation in question.

The compounds of the present invention can be used in combination with other agents useful for treating metabolic and/or eating disorders. The individual components of such combinations can be administered separately at different times during the course of therapy or concurrently in divided or single combination forms. The instant invention is therefore to be understood as embracing all such regimes of simultaneous or alternating treatment and the term “administering” is to be interpreted accordingly. It will be understood that the scope of combinations of the compounds of this invention with other agents useful for treating metabolic and/or eating disorders includes in principle any combination with any pharmaceutical composition useful for treating metabolic and/or eating disorders.

A therapeutically effective amount of a compound of formula (I), or a pharmaceutically acceptable salt or solvate thereof will depend upon a number of factors including, for example, the age and weight of the human or other animal, the precise condition requiring treatment and its severity, the nature of the formulation, and the route of administration, and will ultimately be at the discretion of the attendant physician or veterinarian. However, an effective amount of a compound of formula (I) for the treatment of disorders mediated by the NPY Y5 receptor will generally be in the range of 0.1 to 100 mg/kg body weight of recipient (mammal) per day and more usually in the range of 1 to 10 mg/kg body weight per day. Thus, for a 70 kg adult mammal, the actual amount per day would usually be from 70 to 700 mg and this amount may be given in a single dose per day or more usually in a number (such as two, three, four, five or six) of sub-doses per day such that the total daily dose is the same. An effective amount of a pharmaceutically acceptable salt or solvate thereof, may be determined as a proportion of the effective amount of the compound of formula (I) per se.

A compound of formula (I), or a pharmaceutically acceptable salt or solvate thereof for use in the instant invention may be used in combination with one or more other therapeutic agents. The invention thus provides in a further embodiment a combination comprising a compound of formula (I), or a pharmaceutically acceptable salt or solvate thereof together with a further therapeutic agent, which may be for example an additional anti-obesity agent. In a yet further embodiment the invention also provides the use of a combination comprising a compound of formula (I), or a pharmaceutically acceptable salt or solvate thereof with a further therapeutic agent in the treatment of disorders mediated by the NPY Y5 receptor.

When a compound of formula (I), or a pharmaceutically acceptable salt or solvate thereof is used in combination with one or more other therapeutic agents, the compounds may be administered either sequentially or simultaneously by any convenient route.

The combinations referred to above may conveniently be presented for use in the form of a pharmaceutical formulation and thus pharmaceutical formulations comprising a combination as defined above optimally together with a pharmaceutically acceptable carrier or excipient comprise a further embodiment of the invention. The individual components of such combinations may be administered either sequentially or simultaneously in separate or combined pharmaceutical formulations.

When combined in the same formulation it will be appreciated that the two compounds must be stable and compatible with each other and the other components of the formulation and may be formulated for administration. When formulated separately they may be provided in any convenient formulation, conveniently in such a manner as are known for such compounds in the art.

When a compound is used in combination with a second therapeutic agent active against the same disease, the dose of each compound may differ from that when the compound is used alone. Appropriate doses will be readily appreciated by those skilled in the art.

The following Examples describe the laboratory synthesis of specific compounds of the invention and are not meant to limit the scope of the invention in any way with respect to compounds or processes. It is understood that, although specific reagents, solvents, temperatures and time periods are used, there are many possible equivalent alternatives that can be used to produce similar results. This invention is meant to include such equivalents.

### EXPERIMENTAL

The invention is illustrated by the Compounds described below.

### Abbreviations

DMAP 4-dimethylaminopyridine
DIPEA N,N-diisopropylethylamine
TEA triethylamine
TFA trifluoroacetic acid
EtOAc ethyl acetate
EDC.HCl N-(3-dimethylaminopropyl)-N′-ethylcarbodiimide hydrochloride
HOBt.H₂O 1-hydroxybenzyltriazole hydrate
DMSO dimethylsulfoxide
DCM dichloromethane
DMF N,N-dimethylformamide
HATU (O-7-azabenzotriazol-1-yl)-N,N,N′,N′-tetramethyluroniumhexafluoro-phosphate)
THF tetrahydrofuran
MDAP mass-directed autopurification

Compounds were named using ACD/Name PRO6.02 chemical naming software (Advanced Chemistry Development Inc., Toronto, Ontario, M5H2L3, Canada) with the stereochemical designation (5r,8r) being replaced by the more widely used “trans” designation.

### Analytical Equipment

Proton Magnetic Resonance (NMR) spectra were recorded either on Varian instruments at 300, 400, 500 or 600 MHz, or on Bruker instruments at 300 or 400 MHz. Chemical shifts are reported in ppm (δ) using the residual solvent line as internal standard. Splitting patterns are designated as: s, singlet; d, doublet; t, triplet; q, quartet; m, multiplet; b, broad. The NMR spectra were recorded at a temperature ranging from 25 to 90° C. When more than one conformer was detected the chemical shifts for the most abundant one are reported.

Mass spectra (MS) were taken on a 4 II triple quadrupole Mass Spectrometer (Micromass UK) or on a Agilent MSD 1100 Mass Spectrometer, operating in ES(+) and ES(−) ionization mode. The usage of this methodology is indicated by “MS”. HPLC-Mass spectra (HPLC-MS) were taken on a Agilent LC/MSD 1100 Mass Spectrometer, operating in ES(+) and ES(−) ionization mode coupled with HPLC instrument Agilent 1100 Series [LC/MS-ES (+): analysis performed on a Supelcosil ABZ+Plus (33×4.6 mm, 3 m) (mobile phase: 100% [water+0.1% formic acid] for 1 min, then from 100% [water+0.1% formic acid] to 5% [water+0.1% formic acid] and 95% [acetonitrile] in 5 min, finally under these conditions for 2 min; T=40° C.; flow=1 mL/min; LC/MS-ES (−): analysis performed on a Supelcosil ABZ+Plus (33×4.6 mm, 3 m) (mobile phase: 100% [water+0.05% ammonia] for 1 min, then from 100% [water+0.05% ammonia] to 5% [water+0.05% ammonia] and 95% [acetonitrile] in 5 min, finally under these conditions for 2 min; T=40° C.; flow=1 mL/min]. In the mass spectra only one peak in the molecular ion cluster is reported. The usage of this methodology is indicated by “HPLC-MS 1” in the analytical characterization of the described compounds.

Alternatively, HPLC-MS measurements were carried out using a Platform LCZ™ single quadrupole Mass Spectrometer (Micromass Waters), coupled with an HPLC system Agilent 1100 Series. The experimental conditions were: column XBridge C18, (5 μm 4.6×50 mm), column temperature 30° C., mobile phase, A=water+0.1% TFA and B=MeCN, gradient, t=0 min 0% (B) to 60% (B) in 1.5 min to 95% (B) in 3.5 min lasting for 1.5 min (t=6.60 min 0% B stop time=7.0 min), flow rate 2 ml/min, DAD UV range 210 to 350 nm, MS ionisation mode, positive electrospray (ES+), MS range 110 to 1100 atomic mass unit. The usage of this methodology is indicated by “HPLC-MS 2” in the analytical characterization of the described compounds.

Total ion current (TIC) and DAD UV chromatographic traces together with MS and UV spectra associated with the peaks were taken also on a UPLC-MS Acquity™ system equipped with 2996 PDA detector and coupled to a Waters Micromass ZQ™ mass spectrometer operating in positive or negative electrospray ionisation mode. [LC/MS-ES (+/−): analyses performed using an Acquity™ UPLC BEH C18 column (50×21 mm, 1.7 μm particle size), column temperature 40° C. (mobile phase: A-water+0.1% formic acid/B-acetonitrile+0.075% formic acid, Flow rate: 1.0 mL/min, Gradient: t=0 min 3% B, t=0.05 min 6% B, t=0.57 min 70% B, t=1.4 min 99% B, t=1.45 min 3% B)]. The usage of this methodology is indicated by “UPLC-MS” in the analytic characterization of the described compounds.

For reactions involving microwave irradiation, a Personal Chemistry Emrys™ Optimizer was used.

Flash silica gel chromatography was carried out on silica gel 230-400 mesh (supplied by Merck AG Darmstadt, Germany) or over Varian Mega Be—Si pre-packed cartridges or over pre-packed Biotage silica cartridges.

SPE-SCX cartridges are ion exchange solid phase extraction columns by supplied by Varian. The eluent used with SPE-SCX cartridges is methanol followed by 2N ammonia solution in methanol.

In a number of preparations, purification was performed using either Biotage manual flash chromatography (Flash+) or automatic flash chromatography (Horizon) systems. All these instruments work with standard Biotage Silica cartridges.

SPE-Si cartridges are silica solid phase extraction columns supplied by Varian.

In a number of preparations, purification was performed on a Mass-Directed Autopurification (MDAP) system Fractionlynx™ equipped with Waters 2996 PDA detector and coupled with ZQ™ mass spectrometer (Waters) operating in positive and negative electrospray ionisation mode ES+, ES−. (mass range 100-1000) A set of acidic as well as basic semi-preparative gradients have been used:

METHOD A: Chromatographic Acidic conditions for up to 30 mg of crude:

Column: 100×21.2 mm Supelcosil™ ABZ+Plus (5 μm particle size)

Mobile phase: A[water+0.1% formic acid]/B[acetonitrile+0.1% formic acid]

Flow rate: 20 mL/min

Gradient: 5% B for 1 min, 95% B in 9 min, 100% B in 3.5 min

METHOD B: Chromatographic Acidic conditions for up to 100 mg of crude:

Column: 150×30 mm XTerra Prep MS C18 (10 μm particle size)

Mobile phase: A[water+0.1% formic acid]/B [acetonitrile+0.1% formic acid]

Flow rate: 40 mL/min

Gradient: 1% B to 100% B in 7 min lasting for 7.5 min.

METHOD C: Chromatographic Basic conditions for up to 100 mg of crude

Column: 150×30 mm XTerra Prep MS C18 (10 μm particle size)

Mobile phase: A−water+10 mM ammonium carbonate (adjusted to pH 10 with ammonia)/B−acetonitrile

Flow rate: 40 mL/min

Gradient: 10% B for 0.5 min, 95% B in 12.5 min

All reactions were monitored by thin-layer chromatography on 0.25 mm E. Merck silica gel plates (60E-254), visualised with UV light, iodine, 5% ethanolic phosphomolybdic acid, ninhydrin solution or vanillin solution.

### Supporting Compounds and Intermediates

### Intermediate 1

### Phenyl [4-(2-pyridinyl)-1,3-thiazol-2-yl]carbamate

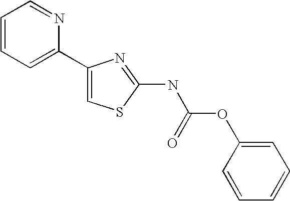

Under a nitrogen atmosphere, 4-(2-pyridinyl)-1,3-thiazol-2-amine (4 g, 0.023 mol, Fluorochem Ltd.) was dissolved in dry pyridine (60 mL) at r.t. The resulting solution was cooled to −10 to −15° C. and phenyl chloroformate (3.4 mL, 0.0271 mol, Aldrich) was added dropwise over 15 min. The reaction was left to stir at this temperature for 1 h, then was quenched at −10° C. by dilution with ethyl acetate and addition of saturated aqueous ammonium chloride solution. The separated organic phase was washed twice with saturated aqueous ammonium chloride solution and dried over anhydrous sodium sulfate. The solvent was evaporated under vacuum to give a crude solid which was purified by two subsequent triturations in diethyl ether. Final filtration of the solid, washing with diethyl ether and drying under vacuum, afforded the title compound (4.54 g, recovery: 67.7%) as an off-white solid;

¹H NMR (600 MHz, DMSO-d6): δ 12.49 (1H, s), 8.57 (1H, d), 7.94 (1H, d), 7.89 (1H, t), 7.85 (1H, m) 7.44 (2H, t), 7.31 (m, 2H), 7.26 (2H, m).

### Intermediate 2

### 3-(3,4-Dichlorophenyl)-1-oxa-3,8-diazaspiro[4.5]decan-2-one

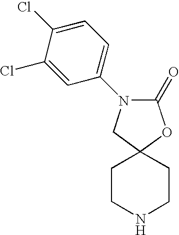

To a suspension of phenylmethyl 3-(3,4-dichlorophenyl)-2-oxo-1-oxa-3,8-diazaspiro[4.5]decane-8-carboxylate (Intermediate 3, 29.58 g, 68 mmol) in industrial methylated spirits (250 ml) was added palladium on carbon (10%, 5 g). The mixture was hydrogenated for 6 h at 50 p.s.i. The mixture was filtered through a celite pad, washing with hot methanol (3×500 ml). The combined filtrates were concentrated under vacuum. The above procedure was repeated; the 2 batches of crude product were combined with another product batch prepared in an analogous manner using phenylmethyl 3-(3,4-dichlorophenyl)-2-oxo-1-oxa-3,8-diazaspiro[4.5]decane-8-carboxylate (2.15 g), industrial methylated spirits (40 ml) and palladium on carbon (10%, 350 mg). The combined batches were recrystallised from methanol to give the title compound (21.97 g);

¹H NMR (DMSO-d6): δ 1.80-1.95 (4H, m), 2.80-3.00 (4H, m), 3.90 (2H, s), 7.55-7.62 (1H, m), 7.63-7.70 (1H, m) and 7.85 (1H, d); m/z 342, [M+MeCN]+.

### Intermediate 3

### Phenylmethyl 3-(3,4-dichlorophenyl-2-oxo-1-oxa-3,8-diazaspiro[4.5]decane-8-carboxylate

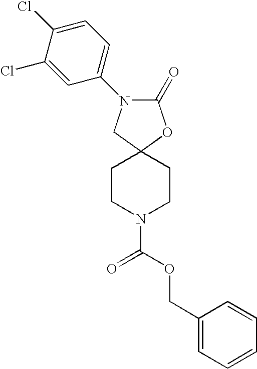

To a stirred solution of phenylmethyl 4-{[(3,4-dichlorophenyl)amino]methyl}-4-hydroxy-1-piperidinecarboxylate (Intermediate 4, 74.9 g, 188 mmol) in dichloromethane (900 ml) was added triethylamine (52.3 ml, 376 mmol). The mixture was cooled to 5° C. (ice bath). To this mixture was added a solution of triphosgene (28.215 g, 95 mmol) in dichloromethane (100 ml) over 20 minutes. The mixture was stirred 1 h 30 min at room temperature, then cooled to 5° C. (ice bath). To the mixture was added aqueous sodium carbonate solution (10%, 250 ml). The organic phase was separated and the aqueous was extracted with dichloromethane (3×50 ml). The combined organic layers were dried over anhydrous magnesium sulfate and concentrated under vacuum to give an orange-brown oil (87.2 g), which crystallised on standing for 48 h. Ethyl acetate (250 ml) was added, and the resultant mixture was heated gently while agitating with a spatula. To the mixture was added hexane (700 ml); the resultant precipitate was filtered off, washed with pentane (2×100 ml) and dried overnight at 50° C. on a hot plate to give the title compound (64.44 g, 78%) as a yellowish solid; Rf 0.36 (1:1 EtOAc—hexane, black spot with phosphomolybdic acid); m.p. 105 C.

### Intermediate 4

### Phenylmethyl 4-{[(3,4-dichlorophenyl)amino]methyl}-4-hydroxy-1-piperidinecarboxylate

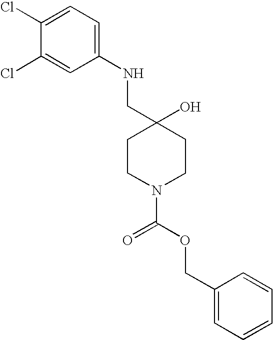

A mixture of phenylmethyl 1-oxa-6-azaspiro[2.5]octane-6-carboxylate (prepared as described in U.S. Pat. No. 4,244,961, 46.6 g, 0.188 mol), 3,4-dichloroaniline (213.4 g, 1.316 mol, Aldrich) and ethoxyethanol (300 ml) was heated under reflux overnight. The mixture was concentrated under vacuum to give the title compound (74.9 g), which was used without further purification; Rf 0.28 (1:1 EtOAc-hexane);

m.p. 128-130 C.

### Intermediate 5

### 2-Oxo-3-phenyl-1-oxa-3-azaspiro[4.5]decane-8-carboxylic acid

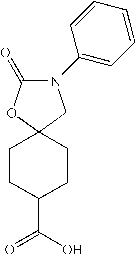

Ethyl 2-oxo-3-phenyl-1-oxa-3-azaspiro[4.5]decane-8-carboxylate (Intermediate 6) (93.2 mg, 0.307 mmol) was dissolved in MeOH (8 ml) and a solution of lithium hydroxide hydrate (63.14 mg) in H₂O (2 ml) was added. The reaction was stirred at r.t. for 4.5 hours. The reaction was acidified with HCl (3N) and then was extracted with EtOAc; the organic phase was dried on Na₂SO₄, filtered and concentrated under vacuum to give the title compound (57.2 mg, 67%);

MS, m/z 276 [M+H]⁺.

A sample prepared using an analogous method showed the followed NMR spectra:

¹H NMR (400 MHz, CDCl₃): δ 7.55 (1H, d), 7.34-7.41 (1H, m), 7.21-7.28 (1H, m), 7.11-7.19 (1H, m), 6.84-7.01 (1H, m), 3.72-3.83 (1H, m), 2.54 (1H, brs), 2.36-2.49 (1H, m H), 0.78-2.21 (9H, m). cis/trans 70:30

### Intermediate 6

### Ethyl 2-oxo-3-phenyl-1-oxa-3-azaspiro[4.5]decane-8-carboxylate

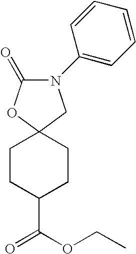

To a stirred solution of ethyl-4-hydroxy-4-({phenyl[(phenyloxy)carbonyl]amino}methyl)-cyclohexanecarboxylate (Intermediate 7) (127.3 mg, 0.320 mmol) in anhydrous toluene (2 ml) was added sodium hydride (60%, 19.21 mg). The reaction was stirred at room temperature overnight. The mixture was poured into water and extracted with EtOAc; the organic phase was dried on Na₂SO₄, filtered and evaporated in vacuo to give crude ethyl 2-oxo-3-phenyl-1-oxa-3-azaspiro[4.5]decane-8-carboxylate (93.2 mg), which was used without further purification; MS, m/z: 304 [M+H]⁺.

Another sample prepared using an analogous method showed the following NMR spectra

¹H NMR (400 MHz, CDCl₃): δ 7.50-7.59 (2H, m), 7.35-7.43 (2H, m), 7.11-7.18 (1H, m), 4.11-4.22 (2H, m), 3.71-3.74 (2H, m), 2.32-2.41 (1H, m), 1.86-2.21 (6H, m), 1.55-1.69 (2H, m), 1.23-1.33 (3H, m). cis/trans 70:30

### Intermediate 7

### Ethyl 4-hydroxy-4-({phenyl[phenyloxy)carbonyl]amino}methyl)cyclohexanecarboxylate

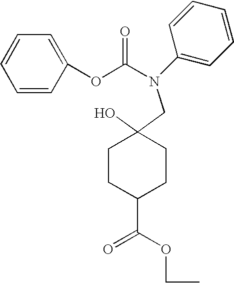

To a stirred solution of crude ethyl 4-hydroxy-4-[(phenylamino)methyl]cyclohexane-carboxylate (Intermediate 8) (3.82 mmol) in DCM (10 ml) at 0° C. were added DIPEA (665 μl, 3.82 mmol) and phenyl chloroformate (480 μl, 3.82 mmol). The reaction was stirred at room temperature overnight. The mixture was poured into a saturated aqueous solution of NH₄Cl and extracted with DCM; the organic phase was dried on Na₂SO₄, filtered and evaporated in vacuo. The crude was purified by silica gel chromatography eluting with cyclohexane/EtOAc to give the title compound (127.3 mg, 8%); (Rf=0.48, cyclohexane/EtOAc 7:3); MS: m/z 398[M+H]⁺. Another batch of the same compound was prepared using an analogous method showed the following NMR spectra: ¹H NMR (400 MHz, CDCl₃): δ 7.28-7.44 (6H, m), 7.16-7.24 (1H, m), 7.03-7.14 (2H, m), 4.05-4.19 (2H, m), 3.91-3.95 (1H, m), 3.82-3.90 (1H, m), 2.39-2.46 (1H, m), 2.14-2.22 (1H, m), 1.47-1.94 (7H, m), 1.18-1.36 (5H, m). cis/trans 70:30

### Intermediate 8

### Ethyl 4-hydroxy-4-[(phenylamino)methyl]cyclohexanecarboxylate

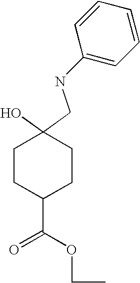

Ethyl 1-oxaspiro[2.5]octane-6-carboxylate (Intermediate 9 procedure 9a, 704.5 mg, 3.82 mmol) was dissolved in t-BuOH (4 ml) and aniline (697 μl, 7.65 mmol, Aldrich) was added. The reaction was stirred and heated at 150° C. under microwave irradiation for two 30 minute cycles. The mixture was poured into a saturated aqueous solution of NH₄Cl and extracted with ethyl acetate; the organic phase was dried on Na₂SO₄, filtered and evaporated in vacuo to give crude ethyl 4-hydroxy-4-[(phenylamino)methyl]-cyclohexanecarboxylate (1.19 g), which was used without further purification. Another batch of the same compound was prepared using an analogous method showed the following NMR spectra:

¹H NMR (400 MHz, CDCl₃): δ 7.14-7.24 (2H, m), 6.65-6.77 (3H, m), 4.10-4.20 (2H, m), 3.16-3.21 (1H, m), 3.09-3.13 (1H, m), 2.45-2.54 (1H, m), 2.23-2.34 (1H, m), 1.36-2.02 (9H, m), 1.22-1.30 (3H, m). cis/trans 65:35

### Intermediate 9

### Ethyl 1-oxaspiro[2,5]octane-6-carboxylate

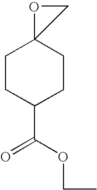

### Procedure 9a

To a mixture of trimethylsulfoxonium iodide and potassium tert-butoxide (as reported in Synthetic Communications, 33(12), 2135-2143; 3.9 g, 11.76 mmol) was added a solution of ethyl 4-oxocyclohexanecarboxylate (1 g, 5.87 mmol, Aldrich) in DMSO (20 ml). The mixture was left to stir overnight at room temperature. The mixture was poured into water and extracted with diethyl ether; the organic phase was dried on Na₂SO₄, filtered and evaporated in vacuo to afford the title compound (704.5 mg, 65%), which was used without purification.

Another batch of the same compound prepared using an analogous method showed the following NMR spectra:

¹H NMR (400 MHz, CDCl3): δ 4.06 (2H, q), 2.49-2.59 (2H, m), 2.26-2.28 (1H, m), 1.63-2.04 (6H, m), 1.27-1.49 (2H, m), 1.20 (3H, t) cis/trans 65:35

### Procedure 9b

A mixture of 2,8,9-thisobutyl-2,5,8,9-tetraaza-1-phosphabicyclo[3.3.3]undecane (1.14 ml, 3.94 mmol) and acetonitrile (15 ml) was added to a stirred suspension of trimethylsulphonium iodide (0.81 g, 3.97 mmol) and ethyl 4-oxocyclohexanecarboxylate (0.563 g, 3.31 mmol) at 0° C. The mixture was stirred at 0° C. for 30 minutes then allowed to warm to room temperature and stirred for a further 1 hour. The reaction mixture was concentrated under reduced pressure then diluted with diethyl ether. The resulting suspension was stirred for 30 minutes then filtered and the filter cake was washed with more diethyl ether. The combined ethereal phases were concentrated under reduced pressure and the residue was chromatographed on SiO₂ (Biotage 25M column) eluting with a gradient of 5%-15% EtOAc/cyclohexane to give a ˜60:40, trans:cis mixture of the title compound as a colourless oil (250 mg);

¹H NMR (400 MHz, CDCl₃): δ 4.16 (2H both isomers, q), 2.65 (2H trans isomer, s), 2.62 (2H cis isomer, s), 2.35-2.48 (1H both isomers, m), 1.68-2.14 (6H both isomers, m), 1.37-1.52 (2H both isomers, m), 1.27 (3H both isomers, t).

### Intermediate 10

### 1,1-Dimethylethyl (trans)-2-oxo-3-phenyl-1-oxa-3-azaspiro[4.5]decane-8-carboxylate

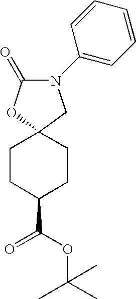

To a stirred mixture of (cis)-2-oxo-3-phenyl-1-oxa-3-azaspiro[4.5]decane-8-carboxylic acid (Intermediate 11, 0.38 g, 1.4 mmol), dimethyl formamide (0.1 ml) and tetrahydrofuran (8 ml) in a round-bottomed flask was added dropwise phosphorus oxychloride (0.15 ml, 1.6 mmol). The mixture was heated to 40° C. and stirred 2 hours. During this time, tetramethyl-ethylenediamine (0.73 ml, 4.8 mmol), tertiary butanol (0.20 ml, 2.1 mmol), lithium chloride (61 mg, 1.4 mmol) and tetrahydrofuran (2 ml) were stirred together in a separate vial. The flask was cooled to room temperature and the contents of the vial were added dropwise to the stirred solution of the intermediate acid chloride in the flask. The mixture was heated to 35° C. and stirred for 18 hours. The mixture was diluted with water and extracted twice with ethyl acetate. The combined organic extracts were washed (water, dilute hydrochloric acid, water), filtered through a hydrophobic membrane and concentrated under vacuum to give the crude product (0.47 g). The crude product was purified by flash column chromatography (silica gel; cyclohexane/ethyl acetate, 10:1); the fractions containing only the faster-running isomer were combined and concentrated under vacuum to give the title compound (0.185 g, 40%) as a viscous oil which crystallised on standing;

¹H NMR (400 MHz, CDCl₃): δ 7.53 (2H, d, J=7.5 Hz), 7.34 (2H, t, J=8 Hz), 7.09 (1H, t, J=7.5 Hz), 3.73 (2H, s), 2.37 (1H, m), 2.08-1.99 (2H, m), 1.96-1.88 (2H, m), 1.87-1.78 (2H, m), 1.72-1.61 (2H, m) and 1.44 (9H, s);

UPLC-MS: 0.85 min, m/z 331 [M+H]+

### Intermediate 11

### (Cis)-2-Oxo-3-phenyl-1-oxa-3-azaspiro[4.5]decane-8-carboxylic acid

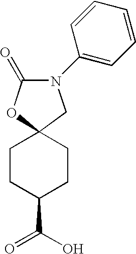

To a stirred solution of ethyl (cis)-2-oxo-3-phenyl-1-oxa-3-azaspiro[4.5]decane-8-carboxylate (prepared in an analogous manner to Intermediate 12, 0.45 g, 1.5 mmol) in methanol (10 ml) was added dropwise a solution of lithium hydroxide (0.18 g) in water (2 ml). The mixture was stirred 1 hour then left to stand for 18 hours. The mixture was acidified with dilute hydrochloric acid (1 M) and extracted twice with ethyl acetate. The combined organic extracts were washed with water, filtered through a hydrophobic membrane and concentrated under vacuum to give the title compound (0.393 g, 96%) as a white solid.

¹H NMR (400 MHz, CDCl₃): δ 7.54 (2H, d, J=7.5 Hz), 7.38 (2H, t, J=7.5 Hz), 7.14 (1H, t, J=7.5 Hz), 3.75 (2H, s), 2.43 (1H, m), 2.19 (1H, m), 2.16 (1H, m), 2.08 (1H, m), 2.06-1.98 (3H, m) and 1.65 (2H, m);

UPLC-MS: 0.62 min, m/z 274 [M−H].

### Intermediates 12 and 13

### Ethyl (cis)-2-oxo-3-phenyl-1-oxa-3-azaspiro[4.5]decane-8-carboxylate (Intermediate 12) and Ethyl (trans)-2-oxo-3-phenyl-1-oxa-3-azaspiro[4.5]decane-8-carboxylate (Intermediate

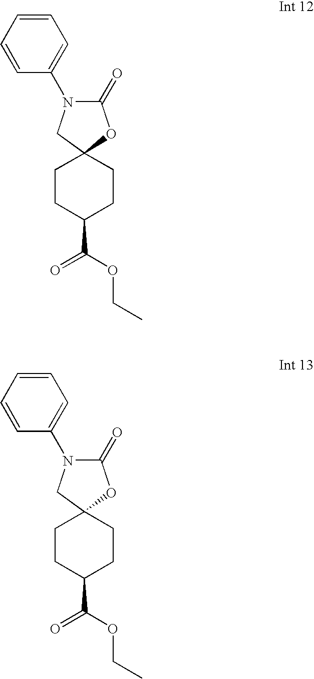

### Procedure 13a

Ethyl 4-hydroxy-4-[(phenylamino)methyl]cyclohexanecarboxylate (prepared in an analogous manner to Intermediate 8, 190.5 mg, 0.68 mmol) was dissolved in anhydrous DCM (10 ml) and was cooled to −50° C. under nitrogen. At this temperature TEA (189.38 μl, 1.36 mmol) and triphosgene (100.691 mg, 0.34 mmol) were added. The reaction was stirred at −78° C. for 2.5 hours. More triphosgene (100.0 mg, 0.337 mmol) was added, and the mixture was stirred for a further 2 hours (until complete). The reaction was treated with a saturated solution of NH₄Cl and was extracted with DCM; the organic phase was dried on Na₂SO₄, filtered and concentrated under vacuum to give a residue (175 mg), which was purified by flash silica gel chromatography (compound Rf=0.27, cyclohexane:EtOAc 7:3). After purification, two separated isomers were obtained: isomer 1 (Intermediate 13, 32.9 mg) and isomer 2 (Intermediate 12, 113.2 mg). The first corresponds to the trans and the second to the cis isomer;

### Isomer 1 (trans), Intermediate 13

¹H NMR (500 MHz, CDCl3): δ 7.55 (2H, d), 7.38 (2H, t), 7.14 (1H, t), 4.16 (2H, q), 3.78 (2H, s), 2.46-2.57 (1H, m), 2.04-2.17 (2H, m), 1.84-2.02 (4H, m), 1.70-1.81 (2H, m), 1.28 (3H, t);

MS: m/z 304 [M+H]+

### Isomer 2 (cis), Intermediate 12

¹H NMR (500 MHz, CDCl3): δ 7.54 (2H, d), 7.38 (2H, t), 7.14 (1H, t), 4.15 (2H, q), 3.74 (2H, s), 2.30-2.42 (1H, m), 2.15 (2H, d), 1.91-2.09 (4H, m), 1.58-1.69 (2H, td), 1.28 (3H, t);

MS: m/z 304[M+H]+.

### Procedure 13b

In a round bottom flask ethyl (trans) 2-oxo-1-oxa-3-azaspiro[4.5]decane-8-carboxylate (Intermediate 15) (0.21 g, 0.924 mmol) was dissolved in toluene (2.1 ml). Iodobenzene (0.207 ml, 1.848 mmol), cesium carbonate (0.753 g, 2.310 mmol), copper(I) iodide (8.80 mg, 0.046 mmol) and trans-1,2-diaminocyclohexane (0.011 ml, 0.092 mmol) were added and the mixture was stirred at 80° C. overnight (overall 24 hours). The mixture was allowed to cool to room temperature and partitioned between water (20 ml) and ethyl acetate (2×20 ml). The combined organics were washed (water), filtered through a Phase Separator filter and concentrated under vacuum.

The crude was purified by column chromatography (silica gel; cyclohexane/ethyl acetate, 1:0 to 10:1 to 6:1, stepped gradient) to give Intermediate 13 (0.165 g, 59%) and Intermediate 12 (0.017 g, 7%).

### Intermediate 13

¹H NMR (400 MHz, CDCl3): δ 7.56 (2H, d), 7.39 (2H, t), 7.15 (1H, t), 4.17 (2H, q), 3.78 (2H, s), 2.48-2.57 (1H, m), 2.07-2.18 (2H, m), 1.85-2.03 (4H, m), 1.70-1.83 (2H, m), 1.29 (3H, t);

UPLC-MS: 0.75 min, m/z 304 [M+H]+.

### Intermediate 12

¹H NMR (400 MHz, CDCl3): δ 7.55 (2H, d), 7.39 (2H, t), 7.15 (1H, t), 4.17 (2H, q), 3.75 (2H, s), 2.32-2.43 (1H, m), 2.12-2-22 (2H, m), 1.90-2.10 (4H, m), 1.58-1.70 (2H, m), 1.29 (3H, t)

UPLC-MS: 0.74 min, m/z 304 [M+H]+.

### Intermediate 14

### (Trans)-2-Oxo-3-phenyl-1-oxa-3-azaspiro[4.5]decane-8-carboxylic acid

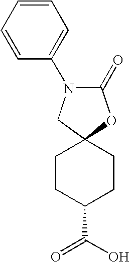

### Procedure 14a

TFA (1.2 ml, 156 mmol) was added to 1,1-dimethylethyl (trans)-2-oxo-3-phenyl-1-oxa-3-azaspiro[4.5]decane-8-carboxylate (prepared in an analogous way to Intermediate 10, 202 mg, 0.61 mmol) in DCM (5.5 ml). The solution was stirred at room temperature for 2 h 30 min, then the mixture was partitioned between DCM and water. The combined organics were washed with water, filtered, dried over Na₂SO₄ and concentrated in vacuum to afford the title compound (170 mg, 0.618 mmol) as a colourless solid.

¹H NMR (400 MHz, CDCl₃): δ 7.50-7.66 (2H, m), 7.32-7.46 (2H, m), 7.09-7.21 (1H, m), 3.71 (2H, s), 2.50-2.74 (1H, m), 2.06-2.27 (2H, m), 1.89-2.01 (4H, m), 1.72-1.89 (2H, m);

UPLC-MS: 0.60 min, m/z 274 [M−H]−.

### Procedure 14b

Ethyl (trans)-2-oxo-3-phenyl-1-oxa-3-azaspiro[4.5]decane-8-carboxylate (this may be prepared as described for Intermediate 13) (260 mg, 0.857 mmol) was dissolved in methanol (5 ml) and water (1 ml) and 5 eq of LiOH (103 mg, 4.29 mmol) were added. The solution obtained was stirred at room temperature for 3 h at which time formation of the acid was complete. Then methanol was evaporated and the crude obtained was partitioned between water and diethylether. The aqueous phase was acidified with HCl 12N until pH 2 was reached and extracted with dichloromethane. The organic phases were collected, dried and then concentrated to afford the title compound (195 mg, 0.708 mmol).

¹H NMR (400 MHz, CDCl₃): δ 7.53-7.58 (2H, m), 7.37-7.43 (2H, m), 7.13-7.19 (1H, m), 3.79 (2H, s), 2.58-2.67 (1H, m), 2.11-2.22 (2H, m), 1.89-2.03 (4H, m), 1.77-1.89 (2H, m).

### Intermediates 15 and 16

Ethyl trans-2-oxo-1-oxa-3-azaspiro[4.5]decane-8-carboxylate (Intermediate 15) and ethyl cis-2-oxo-1-oxa-3-azaspiro[4.5]decane-8-carboxylate (Intermediate 16)

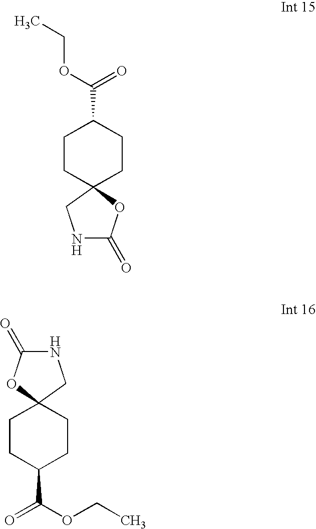

Potassium tert-butoxide (23.14 g, 206 mmol) was added portionwise to a stirred solution of ethyl carbamate (27.6 g, 309 mmol) in DMF (200 ml) at room temperature. The resulting cloudy mixture was stirred for 1 hour then a solution of ethyl 1-oxaspiro[2.5]octane-6-carboxylate (prepared in an analogous manner to intermediate 9 procedure 9b) (19 g, 103 mmol) in DMF (50 ml) was added. The reaction mixture was heated to 130° C. overnight (˜18 hours). Cool and dilute with saturated NaCl solution (20 ml) and extract with AcOEt (4×100 mL). The combined organic layers were dried (Na₂SO₄), filtered and concentrated to a pale yellow oil. The residue was purified via Biotage (cyclohexane:AcOEt starting from 1:1 to AcOEt pure; 65M column) to give Intermediate 15 (8.24 g) and intermediate 16 (4.36 g);

### Intermediate 15

¹H-NMR (400 MHz, CDCl3): δ 5.39 (1H, brs), 4.15 (2H, q), 3.37 (2H, s), 2.47 (1H, sept), 2.01-2.11 (2H, m), 1.80-1.95 (4H, m), 1.62-1.74 (2H, m), 1.27 (3H, t).

### Intermediate 16

¹H-NMR (400 MHz, CDCl3): δ 5.27 (1H, brs), 4.15 (2H, q), 3.32 (2H, s), 2.28-2.37 (1H, m), 2.13 (2H, brd), 1.85-2.05 (4H, m), 1.53 (2H, td), 1.27 (3H, t).

### Intermediate 17

### 5-(2-pyridinyl)-2-pyrazinamine

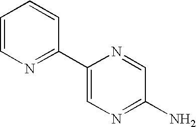

5-Bromo-2-pyrazinamine (commercially available, 250 mg, 1.437 mmol), copper (I) iodide (54.7 mg, 0.287 mmol), bis)triphenylphosphine)palladium(II) chloride (101 mg, 0.144 mmol) and 2-(tributylstannanyl)pyridine (529 mg, 1.437 mmol) were suspended in tetrahydrofuran (15 ml) and stirred at 60° C. for 2 h, then it was warmed to 130° C. under microwave irradiation (2×1 h). The reaction mixture was then rinsed with dichloromethane and washed with water, dried over Na₂SO₄ and concentrated. The resulting crude was purified with Biotage SP1 on KP-NH cartridge, using a gradient of cyclohexane and ethyl acetate. The title compound was eluted with 45% EtOAc and recovered as a colourless solid (90 mg). ¹H NMR (400 MHZ, CDCl₃): δ 9.08 (d, 1H), 8.66 (dq, 1H), 8.17 (dt, 1H), 8.06 (d, 2H), 7.80 (m, 1H), 4.75 (brs, 2H);

UPLC-MS: 0.32 min, 173 [M+H]+.

### Intermediate 18

### 5-(1,3-thiazol-2-yl)-2-pyrazinamine

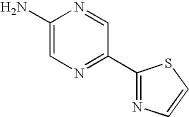

5-bromo-2-pyrazinamine (232 mg, 1.336 mmol), 2-(tributylstannanyl)-1,3-thiazole (500 mg, 1.336 mmol), copper(I)iodide (50.9 mg, 0.297 mmol) and bis(triphenyl-phosphine)palladium(II)chloride (94 mg, 0.134 mmol) were dissolved in THF (14 ml) and stirred at 60° C. for 4.5 h. The mixture was then rinsed with dichloromethane and washed with water. The organic phases were collected, dried and concentrated. The crude obtained was purified on Biotage using a KP-NH 25 M cartridge, eluting with a mixture cyclohexane/EtOAc. The title compound (120 mg, 0.673 mmol) eluted with 30% EtOAc;

¹H NMR (400 MHz, CDCl₃): δ 8.93 (1H, d), 7.98 (1H, d), 7.86 (1H, d), 7.38 (1H, d), 4.95 (2H, brs);

UPLC-MS: 0.45 min, 179 [M+H]+.

### Intermediate 19

### 5-(2-fluorophenyl)-2-pyrazinamine

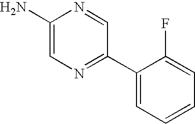

To degassed 1,4-dioxane (17 ml) were added (2-fluorophenyl)boronic acid (0.920 g, 6.57 mmol), 5-bromo-2-pyrazinamine (1.04 g, 5.98 mmol) and bis(triphenylphosphinyl)-palladium(II)chloride (0.210 g, 0.299 mmol). The mixture was stirred at room temperature under nitrogen for 30 minutes. A degassed aqueous solution of sodium carbonate (17.93 ml, 17.93 mmol) was added and the mixture was degassed 3 times (vacuum/nitrogen cycles), heated to reflux and stirred under nitrogen for 3 hours. The mixture was cooled to room temperature and partitioned between water and ethyl acetate. The aqueous was re-extracted with ethyl acetate. The combined organics were washed (water, brine), filtered through a hydrophobic membrane (Phase Separator) and concentrated under vacuum. The crude was purified by column chromatography on silica gel eluting with cyclohexane/ethyl acetate (1:0 to 4:1 to 1:1 gradient then isocratic) to afford the title compound (0.278 g).

¹H NMR (400 MHz, CDCl₃): δ 8.56 (1H, s), 8.12 (1H, d), 7.92 (1H, td), 7.31-7.39 (1H, m), 7.26 (1H, dd), 7.17 (1H, ddd), 4.69 (2H, brs); UPLC-MS: 0.57 min, 190 [M+H]+

A further quantity of title compound (0.555 g) was isolated by recovering an undissolved solid residue from the top of the column, dissolving it in an ethyl acetate/dichloromethane mixture, filtering and concentrating the filtrate under vacuum;

### Intermediate 20

### Ethyl (trans)-2-oxo-3-(2-pyridinyl)-1-oxa-3-azaspiro[4.5]decane-8-carboxylate

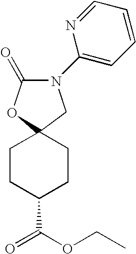

Ethyl (trans)-2-oxo-1-oxa-3-azaspiro[4.5]decane-8-carboxylate (prepared in an analogous manner to Intermediate 15, 700 mg, 3.08 mmol) was dissolved in 7 ml of toluene and 2-iodopyridine (1263 mg, 6.16 mmol), copper(I) iodide (29.3 mg, 0.154 mmol), (+/−)-trans-1,2-diaminocyclohexane (0.037 ml, 0.308 mol) and cesium carbonate (2509 mg, 7.70 mmol) were added and the mixture was heated at 80° C. and stirred vigorously for 18 h under a nitrogen atmosphere in a sealed tube. The mixture was cooled to room temperature and partitioned between water (70 ml) and ethyl acetate (2×100 ml). The combined organic extracts were washed (dilute hydrochloric acid, water), dried over Na₂SO₄, filtered and concentrated under vacuum. The crude was purified with SP1 silica gel column eluting with cyclohexane/ethyl acetate (93:7 to 50:50 gradient) to give the title compound (823.7 mg, 97% yield).

¹H NMR (500 MHz, CDCl₃): δ ppm 8.33 (1H, d), 8.25 (1H, d), 7.68-74 (1H, m), 7.04 (1H, dd), 4.16 (2H, q), 4.03 (2H, s), 2.44-2.52 (1H, m), 2.04-2.15 (2H, m), 1.94-2.02 (2H, m), 1.84-1.92 (2H, m), 1.72-1.83 (2H, m), 1.28 (3H, t); UPLC-MS: 0.74 min, 305[M+H]⁺

### Intermediate 21

### (Trans)-2-oxo-3-(2-pyridinyl)-1-oxa-3-azaspiro[4.5]decane-8-carboxylic acid

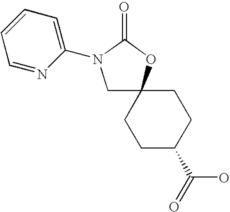

The title compound was made in a similar fashion to the preparation of Intermediate 14 (Procedure 14b) replacing ethyl (trans)-2-oxo-3-phenyl-1-oxa-3-azaspiro[4.5]decane-8-carboxylate with ethyl (trans)-2-oxo-3-(2-pyridinyl)-1-oxa-3-azaspiro[4.5]decane-8-carboxylate (Intermediate 20) to give the title compound (207.8 mg, ˜64% yield);

1H NMR (400 MHz, CDCl3): δ ppm 8.35 (1H, d), 8.29 (1H, dd), 7.69-7.77 (1H, m), 7.01-7.10 (1H, m), 4.05 (1H, s), 4.03 (1H, s), 3.71 (1, s), 3.50 (1H, s), 2.48-2.60 (1H, m), 2.05-2.21 (2H, m), 1.72-2.04 (5H, m).

### Intermediate 22

### Ethyl (trans)-2-oxo-3-(3-pyridinyl)-1-oxa-3-azaspiro[4.5]decane-8-carboxylate

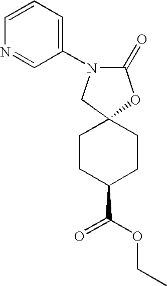

3-bromopyridine (209 mg, 1.320 mmol), ethyl 2-oxo-1-oxa-3-azaspiro[4.5]decane-8-carboxylate (prepared in an analogous fashion to Intermediate 15, 300 mg, 1.320 mmol), trans diaminocyclohexane (15.07 mg, 0.132 mmol) and 1,4-dioxane (20 ml) were collected into a 20 ml microwave vial and stirred at 150° C. for 30 min and then at 160° C. for 2 h30 min under microwave irradiation. The reaction mixture was rinsed with DCM (100 ml) and washed with water (2×20 ml), then dried and concentrated under vacuum. The resulting crude was purified with Biotage SP1, over a 25M KP-NH cartridge, with a gradient of cyclohexane/EtOAc. The title compound was recovered as a colourless solid (190 mg);

¹H NMR (400 MHz, CDCl₃): δ 8.61 (1H, dd), 8.41 (1H, dd), 8.22 (1H, dq), 7.33 (1H, ddd), 4.18 (2H, q), 3.61 (2H, s), 2.50-2.60 (1H, m), 1-75-2.18 (8H, m), 1.29 (3H, t). UPLC-MS: 0.56 min, 305 [M+H]+.

The corresponding cis isomer ethyl (cis)-2-oxo-3-(3-pyridinyl)-1-oxa-3-azaspiro[4.5]-decane-8-carboxylate was also recovered as a colourless solid (120 mg).

¹H NMR (400 MHz, CDCl₃): δ 8.55 (1H, d), 8.40 (1H, dd), 8.25 (1H, dq), 7.32 (1H, ddd), 4.17 (2H, q), 3.71 (2H, s), 2.33-2.43 (1H, m), 2.21-1.93 (6H, m), 1.71-1.61 (2H, m), 1.28 (3H, t).

### Intermediate 23

### Ethyl (trans)-2-oxo-3-(3-pyridazinyl)-1-oxa-3-azaspiro[4.5]decane-8-carboxylate

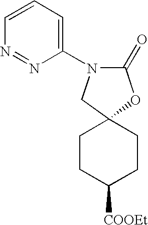

Ethyl (trans)-2-oxo-1-oxa-3-azaspiro[4.5]decane-8-carboxylate (prepared in an analogous fashion to Intermediate 15, 250 mg, 1.100 mmol), 3-chloropyridazine (for a preparation see WO2001007416, 126 mg, 1.100 mmol), trans-1,2-diaminocyclohexane (0.066 ml, 0.550 mmol), copper(I) iodide (105 mg, 0.550 mmol), K₃PO₄ (1168 mg, 5.50 mmol) were collected and shaken at 120° C. for 8 h. Solvent was removed under vacuum, rinsed with DCM (10 ml) and filtered over a separation tube. The resulting solution was then purified with Biotage SP1, over a Silica 25M column, eluting with a gradient of DCM and Et₂O. The title compound was eluted with ca 15% Et₂O and recovered as a colourless solid (110 mg).

¹H NMR (400 MHz, CDCl₃): δ 8.97 (dd, 1H), 8.56 (dd, 1H), 7.50 (dd, 1H), 4.20 (s, 2H), 2.55-2.46 (m, 1H), 2.10-1.74 (m, 8h); UPLC-MS: 0.62 m, 306 [M+H]+.

### Intermediate 24

### (Trans)-2-oxo-3-(3-pyridazinyl)-1-oxa-3-azaspiro[4.5]decane-8-carboxylate lithium salt

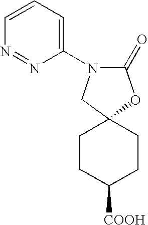

Ethyl (trans)-2-oxo-3-(3-pyridazinyl)-1-oxa-3-azaspiro[4.5]decane-8-carboxylate (Intermediate 23, 115 mg, 0.377 mmol) and lithium hydroxide (0.377 ml, 0.377 mmol) were stirred in methanol (2 ml) over a week. Solvent was then removed under vacuum to afford the title compound (120 mg). This was used in the next step without any further purification.

¹H NMR (400 MHz, CDCl₃): δ 9.00 (dd, 1H), 8.36 (dd, 1H), 7.71 (dd, 1H), 4.06 (1H, s), 3.40 (m, 1H), 2.00-1.50 (m, 8h). UPLC-MS: 0.47 m, 278 [M+H]+.

### EXAMPLES

### Example 1

### Preparation of Compounds of Formula (II)

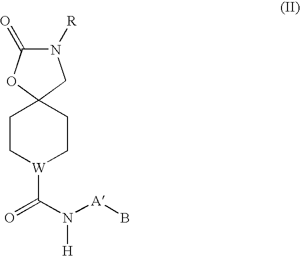

### Example 1-1

### 2-Oxo-3-phenyl-N-[4-(2-pyridinyl)-1,3-thiazol-2-yl]-1-oxa-3,8-diazaspiro[4.5]decane-8-carboxamide hydrochloride

2-Oxo-3-phenyl-N-[4-(2-pyridinyl)-1,3-thiazol-2-yl]-1-oxa-3,8-diazaspiro[4.5]decane-8-carboxamide (Example 1-2, 105 mg, 0.241 mmol) was dissolved in DCM (5 ml) and HCl (1M in Et₂O, 230 μl, 0.228 mmol) was added dropwise at room temperature. The mixture was left for 30 min, then Et₂O was added in order to complete the precipitation. The solvent was removed and the precipitate was dried under vacuum to give the title compound (108 mg, 95%) as a solid;

### Example 1-2

### 2-Oxo-3-phenyl-N-[4-(2-pyridinyl)-1,3-thiazol-2-yl]-1-oxa-3,8-diazaspiro[4.5]decane-8-carboxamide

Phenyl [4-(2-pyridinyl)-1,3-thiazol-2-yl]carbamate (Intermediate 1, 100 mg, 0.336 mmol) was dissolved in CH₃CN (2 ml) and diisopropylethylamine (175.58 μl, 1.008 mmol) and 3-phenyl-1-oxa-3,8-diazaspiro[4.5]decan-2-one (for a preparation see U.S. Pat. No. 4,244,961, 126.21 mg, 0.403 mmol) were added. The mixture was heated at 60° C. for 2.5 hours. The mixture was concentrated under vacuum, and the residue was poured into a saturated solution of NH₄Cl and extracted with DCM. The organic phase was dried over Na₂SO₄, filtered and concentrated under vacuum to give a residue. The residue was purified by silica gel chromatography to give the title compound (106.2 mg, 72%) as a solid;

### Example 1-3

### 3-(3,4-Dichlorophenyl)-2-oxo-N-[4-(2-pyridinyl)-1,3-thiazol-2-yl]-1-oxa-3,8-diazaspiro-[4.5]decane-8-carboxamide

Phenyl [4-(2-pyridinyl)-1,3-thiazol-2-yl]carbamate (Intermediate 1, 50 mg, 0.168 mmol) was dissolved in CH₃CN (1 ml) and diisopropylethylamine (44.24 μl, 0.504 mmol) and 3-(3,4-dichlorophenyl)-1-oxa-3,8-diazaspiro[4.5]decan-2-one (Intermediate 2, 77.10 mg, 0.201 mmol) were added; the mixture was heated at 60° C. for 2.5 hours. The mixture was concentrated under vacuum, and the residue was partitioned between a saturated aqueous solution of NH₄Cl and DCM. The organic phase was dried over Na₂SO₄, filtered and concentrated under vacuum to give a residue. The residue was purified by silica gel chromatography to give the title compound (73.7 mg, 87%) as a solid.

### Example 1-4

### (Trans)-2-Oxo-3-phenyl-N-[4-(2-pyridinyl)-1,3-thiazol-2-yl]-1-oxa-3-azaspiro[4.5]decane-8-carboxamide

2-Oxo-3-phenyl-1-oxa-3-azaspiro[4.5]decane-8-carboxylic acid (Intermediate 5, 29.9 mg, 0.108 mmol) was dissolved in CH₃CN (2 ml) and HOBt.H₂O (16.87 mg, 0.125 mmol) and EDC.HCl (23.96 mg, 0.125 mmol) were added under stirring. After 1.5 hours 4-(2-pyridinyl)-1,3-thiazol-2-amine (16.62 mg, 0.094 mmol, Fluorochem) was added and the reaction was stirred at room temperature for 2 days. The reaction was poured into a saturated solution of NaHCO₃ and extracted with EtOAc; the organic phase was dried on Na₂SO₄, filtered and concentrated under vacuum. The crude (47.6 mg) was purified using MDAP; two separated product isomers were obtained: 10.6 mg of (cis)-2-Oxo-3-phenyl-N-[4-(2-pyridinyl)-1,3-thiazol-2-yl]-1-oxa-3-azaspiro[4.5]decane-8-carboxamide and 5.8 mg of the title compound (trans)-2-oxo-3-phenyl-N-[4-(2-pyridinyl)-1,3-thiazol-2-yl]-1-oxa-3-azaspiro[4.5]decane-8-carboxamide (12%);

### Example 1-5

### (Trans)-2-oxo-3-(2-pyridinyl)-N-[4-(2-pyridinyl)-1,3-thiazol-2-yl]-1-oxa-3-azaspiro[4.5]-decane-8-carboxamide

(Trans)-2-oxo-3-(2-pyridinyl)-1-oxa-3-azaspiro[4.5]decane-8-carboxylic acid (this compound may be prepared following the procedure described for Intermediate 21, 33.7 mg, 0.122 mmol) was dissolved in 2 ml of CH₃CN and HOBt (18.67 mg, 0.122 mmol) and EDC (23.37 mg, 0.122 mmol) were added under stirring at r.t. After 10 min 4-(2-pyridinyl)-1,3-thiazol-2-amine (18.79 mg, 0.106 mmol) was added and the reaction was left in these conditions overnight. The crude was poured into a saturated solution of NaHCO₃ and extracted with ethyl acetate. The organic phase was dried over Na₂SO₄ and concentrated under vacuum to give 48 mg of crude which was purified on silica gel chromatography using a 12M column eluted with cyclohexane/AcOEt from 10 to 50% to give the title compound (15.8 mg, 32.5% yield).

All the analytical data are set forth in the following Table 1-1 and in which W, R, A′ and B have the meanings as follows:

TABLE 1-1

| | |
|---|---|
| | (II) |
| 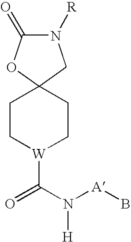 | |

| Cpd. | | | | |
|---|---|---|---|---|
| No. | W | R | A′—B | Analytical Data |
| 1-1 | N | Ph | 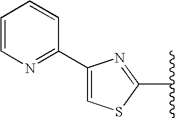 | ¹H NMR (500 MHz, DMSO-d₆): δ 11.37 (1H, br s), 8.71 (1H, d), 8.34 (1H, t), 8.23-8.29 (2H, m), 7.65-7.73 (1H, m), 7.56 (2H, d), 7.39 (2H, t), 7.13 (1H, t), 3.88-3.97 (2H, m), 3.81- 3.89 (2H, m), 3.39-3.55 (2H, m), 1.80-2.01 (4H, m); MS: m/z 436 [M + H]⁺ |
| 1-2 | N | Ph | 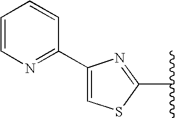 | ¹H NMR (500 MHz, DMSO-d₆): δ 11.15 (1H, s), 8.58 (1H, d), 7.95 (1H, d), 7.86 (1H, t), 7.69 (1H, t), 7.56 (2H, d), 7.39 (2H, t), 7.30 (1H, dd), 7.13 (1H, t), 3.92 (2H, s), 3.81-3.88 (2H, m), 3.44-3.53 (2H, m), 1.85-1.96 (4H, m); MS: m/z 436 [M + H]⁺ |
| 1-3 | N | 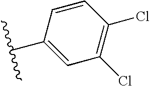 | 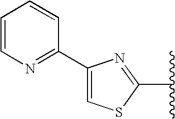 | ¹H NMR (500 MHz, CDCl₃): δ 9.07 (1H, br s), 8.62 (1H, d), 7.80 (1H, d), 7.75 (1H, dt), 7.64-7.67 (1H, m), 7.41-7.49 (3H, m), 7.23 (1H, dd), 4.08 (2H, m), 3.74 (2H, s), 3.47 (2H, t), 2.08 (2H, d), 1.84 (2H, dt); MS: m/z 504 [M + H]⁺ |
| 1-4 | CH | Ph | 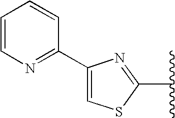 | ¹H NMR (500 MHz, CDCl₃): δ 9.35 (1H, br.s), 8.63 (1H, d), 7.91 (1H, d), 7.77 (1H, t), 7.70 (1H, s), 7.55 (2H, d), 7.39 (2H, t), 7.22-7.26 (1H, dd), 7.15 (1H, t), 3.80 (2H, s), 2.48-2.66 (1H, m), 1.80-2.24 (8H, m); MS: m/z 434 [M + H]⁺ |
| 1-5 | CH | 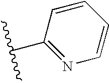 | 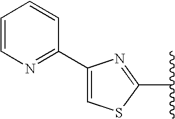 | 1H NMR (500 MHz, CDCl3): δ ppm 9.01 (1H, s), 8.64 (1H, d), 8.34 (1H, d), 8.24 (1H, d), 7.91 (1H, d), 7.77 (1H, t), 7.70-7.74 (1H, m), 7.71 (1H, s), 7.22-7.27 (1H, m), 7.03-7.07 (1H, m), 4.08 (2H, s), 2.51-2.63 (1H, m), 2.14-2.23 (2H, m), 2.07-2.15 (2H, m), 1.88-1.99 (4H, m); UPLC-MS: 0.58 min, 436 [M + H]+. |

### Example 2

### Preparation of Compounds of Formula (IIa)

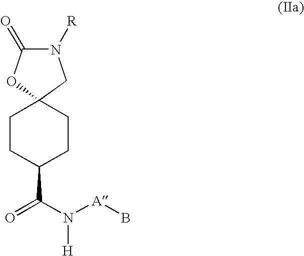

### Example 2-1

### (Trans)-N-[1-(2-Fluorophenyl)-1H-pyrazol-3-yl]-2-oxo-3-phenyl-1-oxa-3-azaspiro[4.5]-decane-8-carboxamide

A solution of (trans)-2-oxo-3-phenyl-1-oxa-3-azaspiro[4.5]decane-8-carboxylic acid (Intermediate 14 procedure 14a, 40 mg, 0.145 mmol), 1-hydroxybenzotriazole hydrate (HOBt.H₂O) (21.55 mg, 0.160 mmol), O-(benzotriazol-1-yl)-tetramethyluronium hexafluorophophosphate (HBTU) (60.7 mg, 0.160 mmol, available on the market) and N-ethyldiisopropylamine (DIPEA) (94 mg, 0.725 mmol) in 2 ml of dichloromethane was stirred at room temperature for 1 h.

Then 1-(2-fluorophenyl)-1H-pyrazole-3-amine (for a preparation see Journal of Organic Chemistry (2005), 70(23), 9222-9229; 28.35 mg, 0.160 mmol) was added and the mixture was stirred at room temperature for 66 h and then left to stand for 96 h. The mixture was then evaporated and the crude was purified using MDAP to afford the title compound (12 mg);

### Example 2-2

### (Trans)-N-[1-(2-fluorophenyl)-1H-pyrazol-3-yl]-2-oxo-3-(3-pyridinyl)-1-oxa-3-azaspiro[4.5]-decane-8-carboxamide hydrochloride

Lithium hydroxide (0.131 ml, 0.131 mmol) was added to a solution of ethyl (trans)-2-oxo-3-(3-pyridinyl)-1-oxa-3-azaspiro[4.5]decane-8-carboxylate (Intermediate 22, 40 mg, 0.131 mmol) in methanol (2 ml). The resulting mixture was stirred at room temperature overnight. Then, solvent was removed and the resulting solid was treated with thionyl chloride (9.59 μl, 0.131 mmol) in N,N-dimethylacetamide (2 ml) at room temperature until complete consumption of starting material. Then pyridine (0.032 ml, 0.394 mmol) was added followed by 1-(2-fluorophenyl)-1H-pyrazol-3-amine (for a preparation see Journal of Organic Chemistry (2005), 70(23), 9222-9229; 25.6 mg, 0.145 mmol). The resulting solution was stirred at room temperature overnight. Then solvent was removed, and the oil was purified by ion exchange cartridge (SCX, 2 g) washing with MeOH and eluting with 2 M Ammonia in MeOH. The resulting crude (50 mg) was then purified with Biotage SP1 on a 12M KP-NH cartridge, with a gradient of cyclohexane/ethyl acetate to afford (trans)-N-[1-(2-fluorophenyl)-1H-pyrazol-3-yl]-2-oxo-3-(3-pyridinyl)-1-oxa-3-azaspiro[4.5]decane-8-carboxamide (14 mg) as colourless solid.

¹H NMR (400 MHz, CDCl₃): δ 8.90 (br s, 1H), 8.34 (brs, 1H), 8.19-8.13 (m, 1H), 8.02 (m, 1H), 7.52-7.47 (m, 3H), 7.61-7.56 (m, 2H), 6.87 (d, 2H), 4.06 (s, 2h), 2.59 (br s, 1H), 2.25-1.74 (dm, 8H); UPLC-MS: 0.60 m, 436 [M+H]+.

(Trans)-N-[1-(2-fluorophenyl)-1H-pyrazol-3-yl]-2-oxo-3-(3-pyridinyl)-1-oxa-3-azaspiro[4.5]decane-8-carboxamide was then dissolved in dichloromethane and treated with 2.0 equivalents of 1M HCl in Et₂O to afford the title compound (13 mg).

### Example 2-3

### (Trans)-N-[1-(2-fluorophenyl)-1H-pyrazol-3-yl]-2-oxo-3-(3-pyridazinyl)-1-oxa-3-azaspiro[4.5]decane-8-carboxamide hydrochloride

(Trans)-2-oxo-3-(3-pyridazinyl)-1-oxa-3-azaspiro[4.5]decane-8-carboxylate lithium salt (Intermediate 24, 50 mg, 0.126 mmol), thionyl chloride (9.21 μl, 0.126 mmol) and N,N-dimethylacetamide (2 ml) were collected and stirred at room temperature. After 24 h there was only trace of the required acid chloride. Solvent and thionyl chloride were removed under vacuum, the residue was triturated with dichloromethane and the resulting crude was suspended in dichloromethane. Oxalxyl chloride (0.017 ml, 0.189 mmol) was added followed by DMF (2 drops), the resulting purple mixture was stirred at room temperature for 2 h. Solvent was removed under vacuum and replaced with N,N-dimethylacetamide (2 ml). Pyridine (0.031 ml, 0.379 mmol) and 1-(2-fluorophenyl)-1H-pyrazol-3-amine (for a preparation see Journal of Organic Chemistry (2005), 70(23), 9222-9229; 22.36 mg, 0.126 mmol) were added and the reaction was stirred overnight at room temperature. Solvent was removed, replaced with dichloromethane (2.0 ml) and HBTU (71.8 mg, 0.189 mmol) and pyridine (0.031 ml, 0.379 mmol) were added. The resulting mixture was stirred overnight then rinsed with dichloromethane (20 ml), washed with water (2×5 ml), and then concentrated. The resulting crude was purified with Biotage SP1, over a 25M KP-NH column, using a gradient of cyclohexane and ethyl acetate. (trans)-N-[1-(2-fluorophenyl)-1-pyrazol-3-yl]-2-oxo-3-(3-pyridazinyl)-1-oxa-3-azaspiro[4.5]decane-8-carboxamide was eluted with 60% EtOAc and recovered as colourless solid (38 mg).

¹H NMR (400 MHz, CDCl₃): δ 8.97 (br, 1H), 8.60-8.47 (m, 2H), 7.95 (br s, 1H), 7.80=7.74 (m, 1H), 7.52-7.44 (m, 1H), 7.28 (br s, 3H), 7.01 (br s, 1H), 4.22 (s, 2H), 2.36 (br s, 1H), 2.18-1.75 (dm, 8H). UPLC-MS: 0.65 m, 437 [M+H]+.

(Trans)-N-[1-(2-fluorophenyl)-1H-pyrazol-3-yl]-2-oxo-3-(3-pyridazinyl)-1-oxa-3-azaspiro[4.5]decane-8-carboxamide was dissolved in dichloromethane (10 ml) and converted into the corresponding hydrochloride salt by means of reaction with 1.0 equiv. of 1M HCl in Et₂O to afford the title compound as colourless solid.

All the analytical data are set forth in the following Table 2-1 and in which R, A″ and B have the meanings as follows:

TABLE 2-1

| | |
|---|---|
| | (IIa) |
| 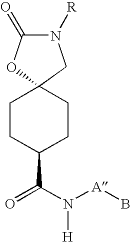 | |

| Cpd. | | | |
|---|---|---|---|
| No. | R | A″—B | Analytical Data |
| 2-1 | Ph | 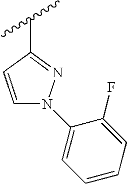 | ¹H NMR: (400 MHz, CDCl₃): δ 8.6 (1H, s), 7.9 (1H, t), 7.85-7.68 (1H, m), 7.63- 7.5 (2H, m), 7.45-7.32 (2H, m), 7.32- 7.20 (4H, m), 7.20-7.09 (1H, m), 7.05- 6.93 (1H, d), 3.86-3.71 (2H, s), 2.45- 2.24 (1H, m), 2.15-2.0 (4H, m), 1.90- 1.66 (4H, m); UPLC-MS: 0.75 min, 435.14 [M + H]+ |
| 2-2 | 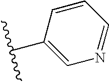 | 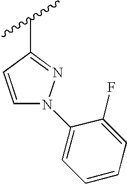 | ¹H NMR (400 MHz, DMSO-d₆): δ 10.75 (s, 1H), 8.97 (s, 1H), 8.49 (br s, 1H), 8.39-8.32 (m, 1H), 8.09 (s, 1H), 7.77- 7.69 (m, 2H), 7.51-7.33 (m, 3H), 6.84 (s, 1H), 4.07 (s, 1H), 2.14-1.60 (dm, 8H); UPLC-MS: 0.60 m, 436 [M + H]+. |
| 2-3 | 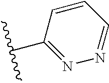 | 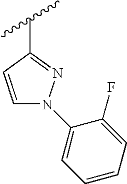 | ¹H NMR (400 MHz, DMSO-d₆): δ 10.76 (br s, 1H), 9.01 (dd, 1H), 8.40 (dd, 1H), 8.10 (t, 1H), 7.77-7.72 (m, 2H), 7.49- 7.33 (m, 2H), 6.85 (d, 1H), 4.19 (s, 1H), 2.60 9m, 1H), 2.15-1.88 (d m, 8h). UPLC-MS: 0.65 m, 4.37 [M + H]+. |

### Example 3

### Preparation of Compounds of Formula (IIb)

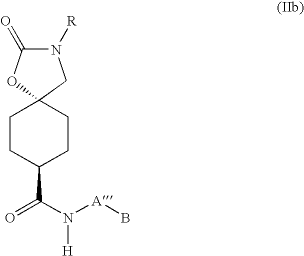

### Example 3-1

### (Trans)-2-Oxo-3-phenyl-N-(5-phenyl-2-pyrazinyl)-1-oxa-3-azaspiro[4.5]decane-8-carboxamide

A mixture of (trans)-2-oxo-3-phenyl-1-oxa-3-azaspiro[4.5]decane-8-carboxylic acid (Intermediate 14 procedure 14a, 40 mg, 0.145 mmol), oxalyl chloride (0.290 mmol) and one drop of DMF in DCM (1 ml) was stirred at room temperature for 3 h 30 min then left to stand for 18 h. The solvent was evaporated under vacuum and the residue obtained was dissolved in 2 ml of toluene. 2-Amino-5-phenylpyrazine (27.4 mg, 0.160 mmol, available on the market.) and DIPEA (56.11 mg, 0.435 mmol) were added to the solution and the mixture was stirred at 90° C. for 3 h and then left to stand for 66 h. The mixture was concentrated under vacuum, then partitioned between DCM and water, neutralized with NaHCO₃ and the combined organic phases were dried over Na₂SO₄ and evaporated under vacuum. The resulting crude was purified by MDAP to afford the title compound (17 mg, 0.040 mmol);

### Example 3-2

### (Trans)-N-(5-bromo-2-pyrazinyl)-2-oxo-3-phenyl-1-oxa-3-azaspiro[4.5]decane-8-carboxamide

One drop of DMF was added to a solution of (trans)-2-oxo-3-phenyl-1-oxa-3-azaspiro[4.5]decane-8-carboxylic acid (prepared in an analogous way to Intermediate 14 procedure 14b, 50 mg, 0.182 mmol) in dichloromethane (3 ml). Oxalyl chloride (34.6 mg, 0.272 mmol) was then added and the resulting mixture was stirred at room temperature for 1 h. Then dichloromethane was evaporated and the crude dissolved in toluene (3.0 ml). DMAP (2.219 mg, 0.018 mmol) and 5-bromo-2-pyrazinamine (37.9 mg, 0.218 mmol) were added and the resulting mixture was stirred at 90° C. for 20 hrs. Toluene was evaporated and the crude obtained was dissolved in dichloromethane and then washed with water. The organic phases were collected, dried and then evaporated. The crude was purified by chromatography with Biotage SP1, on a 12M Silica cartridge, eluting with a mixture cyclohexane/EtOAc. The title compound was recovered impure. The impure compound was triturated with Et₂O to afford the title compound (7 mg).

### Example 3-3

### (Trans)-2-oxo-3-phenyl-N-[5-(1,3-thiazol-2-yl)-2-pyrazinyl]-1-oxa-3-azaspiro[4.5]decane-8-carboxamide hydrochloride

(Trans)-2-oxo-3-phenyl-N-[5-(1,3-thiazol-2-yl)-2-pyrazinyl]-1-oxa-3-azaspiro[4.5]decane-8-carboxamide (15 mg) was prepared in a similar manner to that described for Example 3-2 using 5-(1,3-thiazol-2-yl)-2-pyrazinamine (Intermediate 18) instead of 5-bromo-2-pyrazinamine.

¹H NMR (400 MHz, CDCl₃): δ 9.53 (1H, d), 9.12 (1H, d), 8.22 (1H, brs), 7.95 (1H, d), 7.60-7.55 (2H, m), 7.50 (1H, d), 7.43-7.38 (2H, m), 7.18-7.14 (1H, m), 3.86 (2H, s), 2.62-2.55 (1H, m), 2.55-1.55 (8H, m). UPLC-MS: 0.74 m, 436 [M+H]+

(Trans)-2-oxo-3-phenyl-N-[5-(1,3-thiazol-2-yl)-2-pyrazinyl]-1-oxa-3-azaspiro[4.5]decane-8-carboxamide was dissolved in dichloromethane (2 ml) and treated with 2.1 eq of HCl in diethylether to give the title compound (16 mg).

### Example 3-4

### (Trans)-N-[5-(2-fluorophenyl)-2-pyrazinyl]-2-oxo-3-phenyl-1-oxa-3-azaspiro[4.5]decane-8-carboxamide dihydrochloride

Thionyl chloride (0.013 ml, 0.182 mmol) was added to a solution of (trans)-2-oxo-3-phenyl-1-oxa-3-azaspiro[4.5]decane-8-carboxylic acid (prepared in an analogous way to Intermediate 14 procedure 14b, 50 mg, 0.182 mmol) in N,N-dimethylacetamide (2 ml). The resulting solution was stirred at room temperature for 1 h then a further 1.0 equiv. of SOCl₂ (0.013 ml, 0.182 mmol) was added. After further 1h, 5-(2-fluorophenyl)-2-pyrazin-amine (Intermediate 19, 34.4 mg, 0.182 mmol) and pyridine (0.044 ml, 0.545 mmol) were added. The resulting mixture was stirred overnight. The solvent was then removed under vacuum and the crude was rinsed with dichloromethane (15 ml), washed with water (2 ml), concentrated and purified with Biotage SP1 on a 25M Silica cartridge, using a gradient of dichloromethane and ethanol to afford a mixture of (5r,8r)-N-[5-(2-fluoro-phenyl)-2-pyrazinyl]-2-oxo-3-phenyl-1-oxa-3-azaspiro[4.5]decane-8-carboxamide and 5-(2-fluorophenyl)-2-pyrazinamine as a slightly yellow solid. This mixture was then tritured with Et2O (10×8 ml) to afford (trans)-N-[5-(2-fluorophenyl)-2-pyrazinyl]-2-oxo-3-phenyl-1-oxa-3-azaspiro[4.5]decane-8-carboxamide (44 mg) as a colourless solid.

¹H NMR (400 MHZ, CDCl₃): δ 9.64 (s, 1H), 8.78 (s, 1H), 8.05-8.00 (m, 2H), 7.60-7.5 (2H, m), 7.45-7.40 (3H, m), 7.33-7.14 (m, 3H), 3.86 (s, 2H), 2.57 (br s, 1H), 2.25-1.86 (8H, m). UPLC-MS: 0.81 m, 447 [M+H]+.

(Trans)-N-[5-(2-fluorophenyl)-2-pyrazinyl]-2-oxo-3-phenyl-1-oxa-3-azaspiro[4.5]decane-8-carboxamide was then dissolved in dichloromethane (2 ml) and treated with 2.0 equiv. of 1M HCl in Et₂O to afford the title compound.

### Example 3-5

### (Trans)-2-oxo-3-phenyl-N-[5-(2-pyridinyl)-2-pyrazinyl]-1-oxa-3-azaspiro[4.5]decane-8-carboxamide

(Trans)-2-oxo-3-phenyl-1-oxa-3-azaspiro[4.5]decane-8-carboxylic acid (prepared in an analogous way to Intermediate 14 procedure 14b, 48.0 mg, 0.174 mmol) was suspended in dichloromethane (2 ml). Oxalyl chloride (0.023 ml, 0.261 mmol) was added followed by DMF (8.99 μl, 0.116 mmol) The resulting mixture was stirred at room temperature for 1 h. Solvent was then removed under vacuum rinsed with toluene (2 ml). 5-(2-pyridinyl)-2-pyrazinamine (this may be prepared as described for Intermediate 17) (20 mg, 0.116 mmol) and DMAP (1.419 mg, 0.012 mmol) were added and the mixture was shaken at 90° C. overnight. Solvent was then removed and rinsed with dichloromethane (10 ml) washing with water (2×2 ml). The resulting organic phase was concentrated under vacuum and then purified with Biotage SP1, on a 12M Silica cartridge, using a gradient of dichloromethane/MeOH as eluent to afford a mixture of (trans)-2-oxo-3-phenyl-N-[5-(2-pyridinyl)-2-pyrazinyl]-1-oxa-3-azaspiro[4.5]decane-8-carboxamide and (cis)-2-oxo-3-phenyl-N-[5-(2-pyridinyl)-2-pyrazinyl]-1-oxa-3-azaspiro[4.5]decane-8-carboxamide (85:15 by UPLC-MS, 26 mg) as a yellow solid. This mixture was dissolved in 0.5 ml DMSO and purified by MDAP to afford the title compound (4 mg).

All the analytical data are set forth in the following Table 3-1 and in which R, A′″ and B have the meanings as follows:

TABLE 3-1

| | |
|---|---|
| | (IIb) |
| 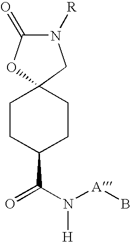 | |

| Cpd. | | | |
|---|---|---|---|
| No. | R | A′′′—B | Analytical Data |
| 3-1 | Ph | 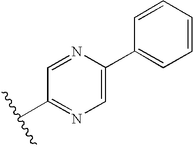 | ¹H NMR: (400 MHz, CDCl₃): δ 9.67 (1H, brs), 8.76 (1H, brs), 7.96-8.07 (2H, m), 7.84-7.96 (1H, brs), 7.54-7.67 (2H, m), 7.45-7.54 (2H, m), 7.33-7.45 (2H, m), 7.05-7.23 (1H, m), 3.79-3.42 (2H, s), 2.43-2.67 (1H, m), 2.06-2.26 (4H, m), 1.78-2.05 (4H, m); HPLC-MS 1: 2.7 min, 429 [M + H]+ |
| 3-2 | Ph | 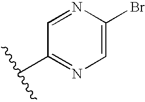 | ¹H NMR: (400 MHz, CDCl₃): δ 9.35 (d, 1H), 8.37 (d, 1H), 7.90 (br s, 1H), 7.60- 7.55 (m, 2H), 7.45-7.35 (m, 2H), 7.20- 7.15 (m, 1H), 3.85 (s, 1H), 2.60-2.63 (m, 1H), 2.26-1.85 (m, 8H); UPLC-MS: 0.76 min, 431 and 433 [M + H]+. |
| 3-3 | Ph | 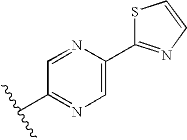 | ¹H NMR (400 MHz, DMSO-d₆): δ 11.10 (1H, brs), 9.38 (1H, d), 9.08 (1H, d), 8.03 (1H, d), 7.92 (1H, d), 7.64 (2H, m), 7.32-7.44 (2H, m), 7.08-7.17 (1H, m), 4.10 (brs), 3.98 (2H, s), 2.67 (br s, 1H), 1.89-2.10 (4H, m), 1.59-1.78 (4H, m); UPLC-MS: 0.74 min, 436 [M + H]+. |
| 3-4 | Ph | 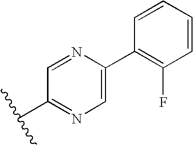 | ¹H NMR (400 MHZ, DMSO-d₆): δ 10.97 (1H, s), 9.48 (1H, s), 8.80 (brs, 1H), 7.98-7.93 (m, 1H), 7.66-7.63 (m, 2H), 7.55-7.50 (m, 1H), 7.42-7.36 (4H, m), 7.15-7.11 (m, 1H), 3.98 (2H, s), 3.82 (brs), 2.68 (brs, 1H), 2.10-1.95 (m, 4H), 1.75-1.65 (m, 4H); UPLC-MS: 0.81 min, 477 [M + H]+. |
| 3-5 | Ph | 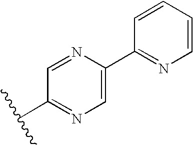 | ¹H NMR (400 MHZ, CDCl₃): δ 9.58 (s, 1H), 9.33 (s, 1H), 8.71 (brs, 1H), 8.34 (d, 1H), 8.07 (s, 1H), 7.86 (t, 1H), 7.59 (d, 2H), 7.42-7.34 (m, 3H), 7.20-7.13 (m, 1H), 3.87 (s, 1H), 2.58 (brs, 1H), 2.28-1.80 (m, 8h); UPLC-MS: 0.71 min, 430 [M + H]+. |

### Example 4

### Preparation of Compounds of Formula (IIc)

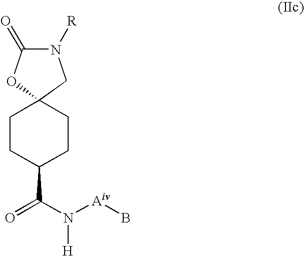

### Example 4-1

### (Trans)-N-[5-(2-Fluorophenyl)-2-pyrimidinyl]-2-oxo-3-phenyl-1-oxa-3-azaspiro[4.5]decane-8-carboxamide

Oxalyl chloride (0.290 mmol) and one drop of DMF were added to (trans)-2-oxo-3-phenyl-1-oxa-3-azaspiro[4.5]decane-8-carboxylic acid (Intermediate 14 procedure 14a, 40 mg, 0.145 mmol) in DCM (1 ml). The mixture was stirred at room temperature for 4 h and then left to stand for 18 h. The solvent was removed under vacuum and the residue obtained was dissolved in 2 ml of toluene. To the resultant solution were added 5-(2-fluorophenyl)-2-pyrimidinamine (for a preparation see WO2003010175, 30.3 mg, 0.160 mmol), DIPEA (56.11 mg, 0.435 mmol) and 4-dimethylaminopyridine (DMAP) (3.5 mg, 0.029 mmol) and the mixture was stirred at 90° C. for 5 h and then left to stand for 18 h. The mixture was concentrated under vacuum; the residue was purified by MDAP to afford the title compound as a solid (8 mg);

All the analytical data are set forth in the following Table 4-1 and in which R, A^{iv} and B have the meanings as follows:

TABLE 4-1

| | |
|---|---|
| | (IIc) |
| 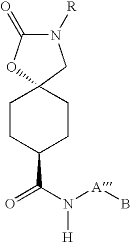 | |

| Cpd. | | | |
|---|---|---|---|
| No. | R | A^{iv}—B | Analytical Data |
| 4-1 | Ph | 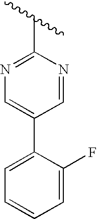 | ¹H NMR (400 MHz, CDCl₃): δ 8.94-8.71 (2H, d), 8.55 (1H, brs), 7.50-7.64 (2H, m), 7.50-7.36 (3H, m), 7.36-7.19 (3H, m), 7.10-7.19 (1H, m), 3.8 (2H, s), 3.15 (1H, brs), 2.2-1.7 (8H, m); UPLC-MS: 0.71 min, 447.14 [M + H]+ |

### Example 5

### Preparation of Compounds of Formula (IId)

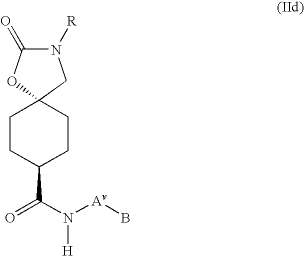

### Example 5-1

### (Trans)-2-oxo-3-phenyl-N-3-quinolinyl-1-oxa-3-azaspiro[4.5]decane-8-carboxamide hydrochloride

(Trans)-2-oxo-3-phenyl-1-oxa-3-azaspiro[4.5]decane-8-carboxylic acid (Intermediate 14 procedure 14b, 0.182 mmol, 50 mg) was dissolved in dimethylacetamide (1 ml) in a nitrogen-purged vial. The solution was stirred, cooled to −10° C. and treated with thionyl chloride (0.191 mmol, 14 μl) over 30 min. The complete formation of the acyl chloride was monitored via mass spectrometry. Then a previously prepared solution of 3-quinolinamine (0.193 mmol, 27.8 mg) in dimethylacetamide (1 ml) and pyridine (0.5 ml) was added and the reaction mixture was left to warm to room temperature and stirred for 42 h. The solution was loaded onto an SCX cartridge and eluted at first with methanol and then with a solution 2M of ammonia in methanol. The title compound was not purified from the starting amine so the two aliquots were added together and the solvents were evaporated. The resulting crude was partitioned between dichloromethane and water. The organic phases were collected and the solvent evaporated. The resulting crude was purified with silica column cromatography eluting with a gradient of cyclohexane/EtOAc. The product was purified at 100% of EtOAc obtaining (trans)-2-oxo-3-phenyl-N-3-quinolinyl-1-oxa-3-azaspiro[4.5]decane-8-carboxamide (31 mg, 0.077 mmol).

¹H NMR (400 MHz, CDCl3) δ 8.78-8.89 (2H, m), 8.52 (1H, s), 8.06 (1H, d), 7.81 (1H, d), 7.62-7.69 (1H, m), 7.53-7.61 (3H, m), 7.41 (2H, t), 7.17 (1H, t), 3.88 (2H, s), 2.56-2.65 (1H, m), 2.13-2.23 (4H, m), 1.83-1.98 (4H, m).

HPLC/MS: 2.33 min, 402 [M+H]+

The hydrochloride salt was prepared by dissolving (trans)-2-oxo-3-phenyl-N-3-quinolinyl-1-oxa-3-azaspiro[4.5]decane-8-carboxamide (31 mg, 0.077 mmol) in 2 ml of dichloromethane, adding HCl in diethylether 1M (0.1 ml, 0.1 mmol) and stirring it for half an hour. Dichloromethane was evaporated and the crude obtained was triturated in diethylether to afford the title compound (20 mg, 0.046 mmol).

### Example 5-2

### (Trans)-2-oxo-3-(2-pyridinyl)-N-3-quinolinyl-1-oxa-3-azaspiro[4.5]decane-8-carboxamide dihydrochloride

(Trans)-2-oxo-3-(2-pyridinyl)-1-oxa-3-azaspiro[4.5]decane-8-carboxylic acid (Intermediate 21, 155.3 mg, 0.562 mmol) was dissolved in 1,2-dichloroethane (4 ml) and thionyl chloride (0.041 ml, 0.562 mmol) was added at r.t under stirring for 2 h. The reaction mixture was concentrated under reduced pressure to give (160 mg, quantitative yield) of crude trans-2-oxo-3-(2-pyridinyl)-1-oxa-3-azaspiro[4.5]decane-8-carbonyl chloride. 54 mg of this crude (0.183 mmol) was dissolved in 1,2-dichloroethane (3 ml) and 3-quinolinamine (26 mg, 0.183 mmol) and DIPEA (0.038 ml, 0.220 mmol) were added, the reaction was heated at 90° C. overnight. The reaction was poured into a solution of NH4Cl saturated solution and extracted with DCM, the organic phase was dried over Na2SO4 and concentrated under vacuum. The crude was purified on SP1 on a NH cartridge eluting with DCM:Et₂O to give (8.8 mg, 12% yield) of (trans)-2-oxo-3-(2-pyridinyl)-N-3-quinolinyl-1-oxa-3-azaspiro-[4.5]decane-8-carboxamide.

¹H NMR (400 MHz, CDCl3): δ ppm 8.86 (1H, dd), 8.41 (1H, d), 8.31-8.36 (1H, m), 8.20-8.28 (1H, m), 8.15 (1H, d), 8.07 (1H, d), 7.69-7.76 (1H, m), 7.49-7.65 (1H, m), 7.41 (1H, dd), 7.01-7.08 (1H, m), 5.17-5.45 (1H, m), 4.08-4.16 (2H, m), 3.63-3.69 (2H, m), 3.34-3.49 (1H, m), 2.46-2.61 (1H, m), 2.07-2.29 (2H, m), 1.96 (3H, s).

To a solution of (trans)-2-oxo-3-(2-pyridinyl)-N-3-quinolinyl-1-oxa-3-azaspiro[4.5]decane-8-carboxamide (8.8 mg, 0.022 mmol) in DCM (2 ml) was added drop by drop under stirring a solution of HCl in Et2O (1 M 0.048 ml, 0.048 mmol). The solution was left at r.t. under stirring for 30 minutes and then the precipitate was separated, triturated with Et2O, dried under a flow of nitrogen and then for 18 h under high vacuum at 40° C. to give the title compound (7.3 mg, 70% yield);

All the analytical data are set forth in the following Table 5- and in which R, A^{v} and B have the meanings as follows:

TABLE 5-1

| | |
|---|---|
| | (IId) |
| 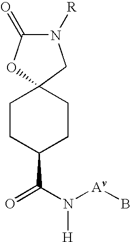 | |

| Cpd. | | | |
|---|---|---|---|
| No. | R | A^{v}—B | Analytical Data |
| 5-1 | Ph | 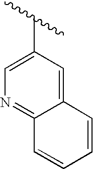 | ¹H NMR (600 MHz, DMSO-d₆) δ 8.95- 9.00 (1 H, m), 8.71-8.78 (1 H, m), 7.91- 7.97 (2 H, m), 7.65 (1 H, t), 7.62 (2 H, d), 7.57 (1 H, t), 7.38 (2 H, t), 7.11 (1 H, t), 3.97 (2 H, s), 2.47-2.60 (1 H, m), 2.01-2.10 (2 H, m), 1.94-2.01 (2 H, m), 1.65-1.80 (4 H, m); UPLC-MS: 0.69 min, 402 [M + H]+ |
| 5-2 | 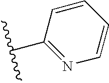 | 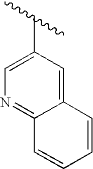 | 1H NMR (500 MHz, MeOH-d₄) δ ppm 9.10 (1H, dd), 8.83 (1H, d), 8.39 (1H, d), 8.19-8.26 (1H, m), 8.12 (1H, d), 8.01-8.08 (2H, m), 7.90-7.95 (1H, m), 7.20-7.29 (2H, m), 4.16-4.19 (2H, m), 3.75 (1H, t), 3.63-3.66 (1H, m), 3.45 (1H, t), 3.17 (1H, t), 2.59-2.67 (1H, m), 2.07-2.21 (4H, m), 1.81-1.97 (3H, m); UPLC-MS: 0.63 min, 403 [M + H]+ |

### Example 6

### Preparation of Compounds of Formula (IIe)

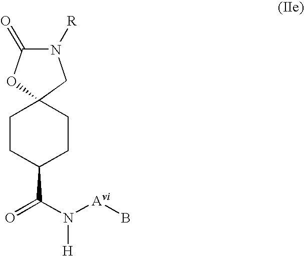

### Example 6-1

### (Trans)-2-oxo-3-phenyl-N-2-quinoxalinyl-1-oxa-3-azaspiro[4.5]decane-8-carboxamide hydrochloride

DIPEA (0.048 mL, 0.272 mmol) and propylphosphonic anhydride (0.106 mL, 0.182 mmol) were added to a solution of (trans)-2-oxo-3-phenyl-1-oxa-3-azaspiro[4.5]decane-8-carboxylic acid (prepared in an analogous way to Intermediate 14 procedure 14b, 50 mg, 0.182 mmol) in 1,4-dioxane (1.5 ml) and stirred at room temperature for 1.5 h. Then 2-quinoxalinamine (26.4 mg, 0.182 mmol) in 1,4-dioxane (1 ml) was added and the resulting solution was stirred at 80° C. for 20 hrs. Solvent was evaporated and the crude obtained dissolved in dichloromethane and washed twice with H₂O. The organic phases were collected, dried over Na₂SO₄ and the dichloromethane was evaporated. The crude obtained was purified with Biotage SP1, over a 12M Silica cartridge, eluting with a mixture cyclohexane/EtOAc. (trans)-2-oxo-3-phenyl-N-2-quinoxalinyl-1-oxa-3-azaspiro[4.5]-decane-8-carboxamide was eluted with 40% EtOAc (4 mg).

¹H NMR (400 MHz, CDCl₃): δ 9.85 (s, 1H), 8.2 (brs, 1H), 8.15-8.10 (m, 1H), 7.87-7.85 (m, 1H), 7.78-7.69 (m, 2H), 7.61-7.56 (m, 2H), 7.44-7.39 (m, 2H), 7.20-7.15 (m, 1H), 3.86 (2H, s), 2.53-2.70 (1H, m), 2.05-2.34 (4H, m), 1.85-2.02 (4H, m).

UPLC-MS: 0.73 min, 403 [M+H]+.

(Trans)-2-oxo-3-phenyl-N-2-quinoxalinyl-1-oxa-3-azaspiro[4.5]decane-8-carboxamide was dissolved in 1 ml of DCM and of 2.1 eq of a solution 1M of HCl in diethylether were added to afford the title compound (4 mg).

All the analytical data are set forth in the following Table 6-1 and in which R, A^{vi} and B have the meanings as follows:

TABLE 6-1

| | |
|---|---|
| | (IIe) |
| 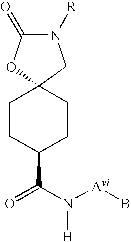 | |

| Cpd. | | | |
|---|---|---|---|
| No. | R | A^{vi}—B | Analytical Data |
| 6-1 | Ph | 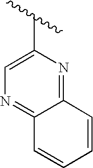 | ¹H NMR (400 MHz, DMSO-d₆): δ 11.16 (1H, s), 9.64 (1H, s), 8.02-8.07 (1H, m), 7.87-7.91 (1H, m), 7.78-7.84 (1H, m), 7.69-7.75 (1H, m), 7.61-7.66 (1H, m), 7.36-7.42 (1H, m), 7.09-7.15 (1H, m), 3.98 (2H, s), 2.64-2.71 (1H, m), 1.94- 2.11 (4H, m), 1.68-1.78 (4H, m). |

### Example 7

### In Vitro Profile

The in vitro assessment of the NPY-Y5 antagonist compounds used different assay systems to determine the potency and affinities against the NPY-Y5 receptor.

The affinities of the compounds of the invention for the NPY Y5 receptor may be determined by the binding assays described below. Such affinity is typically calculated from the IC₅₀ obtained in competition experiments as the concentration of a compound necessary to displace 50% of the radiolabeled ligand from the receptor, and is reported as a “Kᵢ” value calculated by the following equation:

$${K}_{i} = \frac{{IC}_{50}}{1 + L / {K}_{D}}$$

where L=radioligand and K_{D}=affinity of radioligand for receptor (Cheng and Prusoff, Biochem. Pharmacol. 22: 3099, 1973). In the context of the present invention pKi values (corresponding to the antilogarithm of Ki) are used instead of Ki; pKi results are only estimated to be accurate to about 0.3-0.5.

The functional activity of the compounds of the invention for the NPY Y5 receptor may be determined by the FLIPR/Ca²⁺ assay as described below. Such potency is typically calculated from the IC₅₀ obtained in FLIPR experiments as the concentration of a compound necessary to decrease 50% of the calcium release following cells exposure to a concentration of PYY eliciting 80% response (i.e. EC80), and is reported as a “fKᵢ” value calculated by the following equation:

$${fK}_{i} = \frac{{IC}_{50}}{1 + EC80 / EC50}$$

where EC80 and EC50 corresponding to the agonist (PYY) concentrations that eliciting 80% and 50% response, respectively (corresponding to the Cheng and Prusoff equation). In the context of the present invention pfKi values (corresponding to the antilogarithm of fKi) are used instead of fKi; pfKi results are only estimated to be accurate to about 0.3-0.5.

### Functional Activity at Recombinant Human NPY-Y5 Receptor

The functional activity at the human NPY-Y5 receptor stably expressed in HEK293 cells was assessed using FLIPR/Cα²⁺ methodology (cell line name: HEK 293 signal-hNPY-Y5/G16z49). The assay is configured to re-direct receptor-mediated signalling to the calcium release from intracellular stores via the promiscuous Gα16z49 protein. PYY (peptide YY) is an endogenous agonist and can activate the receptor, thereupon causing an increase in the level of calcium in the cells sensed by Fluo-4-AM and measured by FLIPR. Antagonist effects are monitored by the blockade or decrease in calcium release once cells co-expressing hNPY-Y5 receptor and Gα16z49 are exposed to a concentration of PYY eliciting 80% response (i.e. EC80). A non-linear, 4 parameter logistic curve-fit of the data generated pIC₅₀ value. Applying the Cheng-Prusoff equation to antagonist concentration-response for inhibition of fixed PYY concentration yielded the fpKi values.

Cells are cultured in DMEM/F12 supplemented with 10% FBS, 2 mM Glutamine, 200 μg/mL hygromycin B and 500 μg/mL G418. The day before a FLIPR experiment, cells are plated out into 384-well Poly-D-Lysine coated FLIPR plates at a density of 200′000 cells/mL corrects to give 10′000 cells per 50 μL per well using medium without antibiotics.

On the day of experiment, cells are washed with an assay buffer containing 20 mM HEPES/NaOH, 145 mM NaCl, 5 mM KCl, 1 mM MgCl₂, 2 mM CaCl₂, 1 g/L D-glucose and 2.5 mM probenecid, pH 7.3 and loaded with 2 μM Fluo-4 AM for 60 min at 37° C. and 5% CO₂. The excess of dye solution is removed by washing cells with buffer. Compound solutions, prepared by serially diluting compounds in neat DMSO and then a final 1:50 dilution step in assay buffer added with 0.05% pluronic acid, are added and incubated with the loaded cells for 30 min at 37° C. and 5% CO₂. Cells are then put in the FLIPR for the stimulus addition corresponding to a concentration of PYY eliciting 80% of the response. The response of cells to the agonist is fast and measured for 2 min after PYY addition.

### Binding Affinities at Human and rat NPY-Y5 Receptors

The assays used to measure compound affinity to human and rat NPY5 receptors were binding assays using Scintillation Proximity Assay (SPA) technology. The SPA involves the coupling of cell membrane fragments, via their glycosylated residues, to the wheat germ agglutinin (WGA) present on the surface of SPA beads. This coupling mechanism immobilises receptors in close proximity to the scintillant within the SPA beads and binding to the receptors of a radiolabelled ligand can thus be measured directly without the need to separate bound from free ligand.

Binding experiments are carried out in 384-well plates. The assay buffer contains 50 mM HEPES/NaOH pH 7.4, 1 mM MgCl₂, 2.5 mM CaCl₂ and 0.05% pluronic acid. Specific binding is defined as the portion of [125I]-porcinePYY that is displaceable by 1 μM human PYY. A non-linear, 4 parameter logistic curve-fit of the data generated pIC₅₀ and pKi values.

### 125I-PYY Binding on Human NPY-Y5 BacMam Membranes

Competition experiments are carried out in 384-well white with clear bottom plates in a final volume of 50 μL. PVT-WGA beads and membranes (prepared from HEK293F G0 cells) are diluted in assay buffer to have 2.5 mg/mL and 50 μg/mL, respectively and precoupled at 4° C. for 60 min. [125I]-PYY is added to the membrane-beads mix to achieve a concentration of 20 pM. 50 μL of the SPA mix is added to each well containing 0.5 μL compound solution. Compound solutions are prepared by serially diluting compounds in neat DMSO. The incubation lasted 3 hours at room temperature under gentle shaking. Then plates are left overnight at room temperature to allow the beads to settle and bound radioactivity is measured using Trilux MicroBeta.

### 125I-PYY Binding on Rat NPY-Y5 BacMam Membranes

Competition experiments are carried out in 384-well white plates in a final volume of 30 μL. WGA-Polystyrene LEADseeker imaging beads and membranes (prepared from HEK293F G0 cells), are diluted in assay buffer to have 2.5 mg/mL and 30 μg/mL, respectively and precoupled at 4° C. for 60 min. [125]I-PYY is added to the membrane-beads mix to achieve a concentration of 75 pM. 30 μL of the SPA mix is added to each well containing 0.3 μL compounds solution. Compound solutions are prepared by serially diluting compounds in neat DMSO. The incubation lasted 3 hours at room temperature under gentle shaking. Then plates are left overnight at room temperature and bound radioactivity is measured using ViewLux.

The compounds of formula (I) typically show pKi greater than 6 towards NPY Y5 receptor. In one embodiment, the compounds of formula (I) typically show pKi greater than 7 for the NPY Y5 receptor. In another embodiment, the compounds of formula (I) typically show pKi greater than 8 for the NPY Y5 receptor. In a further embodiment, the compounds of formula (I)′ typically show pKi greater than 9 for the NPY Y5 receptor.

1. A compound of formula (I) or a pharmaceutically acceptable salt or solvate thereof, 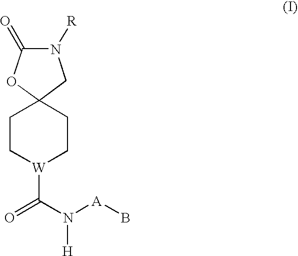
wherein
R is an aryl or heteroaryl; which may be substituted by one or more: halogen, C1-C4 alkyl, C1-C4 alkoxy, C1-C4 haloalkyl, C1-C4 haloalkoxy, cyano;
W is —CZ₁ or nitrogen;
Z₁ is hydrogen, C1-C4 alkyl;
A is a 5 membered heteroaryl, pyrazine, pyrimidine, or quinoline or quinazoline which may be substituted by one or more: halogen, C1-C4 alkyl, C1-C4 alkoxy, C1-C4 haloalkyl, C1-C4 haloalkoxy, cyano;
B is hydrogen or a 5-10 membered aryl or heteroaryl which may be substituted by one or more: halogen, C1-C4 alkyl, C1-C4 alkoxy, C1-C4 haloalkyl, C1-C4 haloalkoxy, cyano; being A and B linked via any atom;
provided that when W is —CZ₁, compounds of formula (I) have a trans stereochemistry.

2. A compound of formula (II) according to claim 1 or a pharmaceutically acceptable salt or solvate thereof, 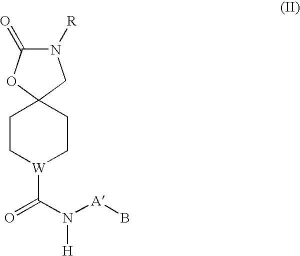
wherein
R is an aryl or heteroaryl; which may be substituted by one or more: halogen, C1-C4 alkyl, C1-C4 alkoxy, C1-C4 haloalkyl, C1-C4 haloalkoxy, cyano;
A′ is a thiazole which may be substituted by one or more: halogen, C1-C4 alkyl, C1-C4 alkoxy, C1-C4 haloalkyl, C1-C4 haloalkoxy, cyano;
W is —CZ₁ or nitrogen;
Z₁ is hydrogen, C1-C4 alkyl;
B is hydrogen or a 5-10 membered aryl or heteroaryl which may be substituted by one or more: halogen, C1-C4 alkyl, C1-C4 alkoxy, C1-C4 haloalkyl, C1-C4 haloalkoxy, cyano;
provided that when W is —CZ₁, compounds of formula (I) have a trans stereochemistry.

3. A compound of formula (IIa) according to claim 1 or a pharmaceutically acceptable salt or solvate thereof, 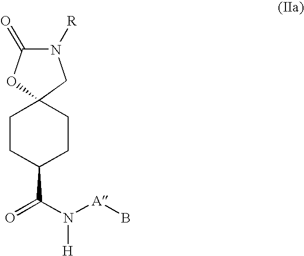
wherein
R is an aryl or heteroaryl; which may be substituted by one or more: halogen, C1-C4 alkyl, C1-C4 alkoxy, C1-C4 haloalkyl, C1-C4 haloalkoxy, cyano;
A″ is a pyrazole which may be substituted by one or more: halogen, C1-C4 alkyl, C1-C4 alkoxy, C1-C4 haloalkyl, C1-C4 haloalkoxy, cyano;
B is hydrogen or a 5-10 membered aryl or heteroaryl which may be substituted by one or more: halogen, C1-C4 alkyl, C1-C4 alkoxy, C1-C4 haloalkyl, C1-C4 haloalkoxy, cyano.

4. A compound of formula (IIb) according to claim 1 or a pharmaceutically acceptable salt or solvate thereof, 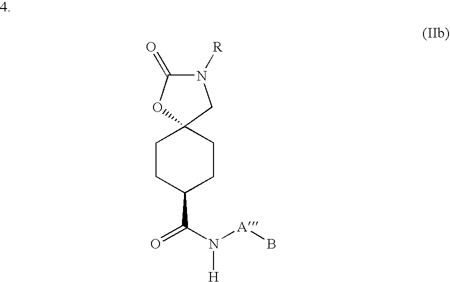
wherein
R is an aryl or heteroaryl; which may be substituted by one or more: halogen, C1-C4 alkyl, C1-C4 alkoxy, C1-C4 haloalkyl, C1-C4 haloalkoxy, cyano;
A′″ is a pyrazine which may be substituted by one or more: halogen, C1-C4 alkyl, C1-C4 alkoxy, C1-C4 haloalkyl, C1-C4 haloalkoxy, cyano;
B is hydrogen or a 5-10 membered aryl or heteroaryl which may be substituted by one or more: halogen, C1-C4 alkyl, C1-C4 alkoxy, C1-C4 haloalkyl, C1-C4 haloalkoxy, cyano.

5. A compound of formula (IIc) according to claim 1 or a pharmaceutically acceptable salt or solvate thereof, 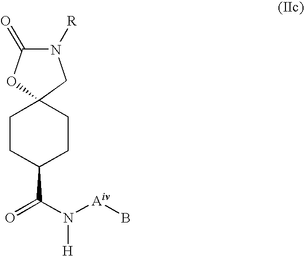
wherein
R is an aryl or heteroaryl; which may be substituted by one or more: halogen, C1-C4 alkyl, C1-C4 alkoxy, C1-C4 haloalkyl, C1-C4 haloalkoxy, cyano;
A^{iv} is a pyrimidine which may be substituted by one or more: halogen, C1-C4 alkyl, C1-C4 alkoxy, C1-C4 haloalkyl, C1-C4 haloalkoxy, cyano;
B is hydrogen or a 5-10 membered aryl or heteroaryl which may be substituted by one or more: halogen, C1-C4 alkyl, C1-C4 alkoxy, C1-C4 haloalkyl, C1-C4 haloalkoxy, cyano.

6. A compound of formula (IId) according to claim 1 or a pharmaceutically acceptable salt or solvate thereof, 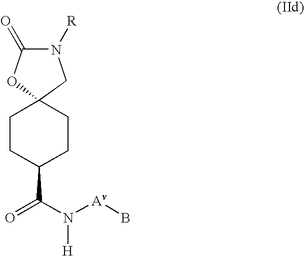
wherein
R is an aryl or heteroaryl; which may be substituted by one or more: halogen, C1-C4 alkyl, C1-C4 alkoxy, C1-C4 haloalkyl, C1-C4 haloalkoxy, cyano;
A^{v} is a quinoline which may be substituted by one or more: halogen, C1-C4 alkyl, C1-C4 alkoxy, C1-C4 haloalkyl, C1-C4 haloalkoxy, cyano;
B is hydrogen or a 5-10 membered aryl or heteroaryl which may be substituted by one or more: halogen, C1-C4 alkyl, C1-C4 alkoxy, C1-C4 haloalkyl, C1-C4 haloalkoxy, cyano.

7. A compound of formula (IIe) according to claim 1 or a pharmaceutically acceptable salt or solvate thereof, 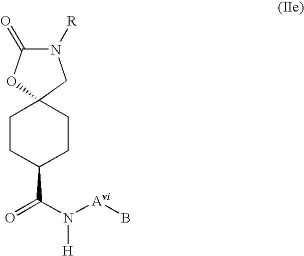
wherein
R is an aryl or heteroaryl; which may be substituted by one or more: halogen, C1-C4 alkyl, C1-C4 alkoxy, C1-C4 haloalkyl, C1-C4 haloalkoxy, cyano;
A^{vi} is a quinazoline which may be substituted by one or more: halogen, C1-C4 alkyl, C1-C4 alkoxy, C1-C4 haloalkyl, C1-C4 haloalkoxy, cyano;
B is hydrogen or a 5-10 membered aryl or heteroaryl which may be substituted by one or more: halogen, C1-C4 alkyl, C1-C4 alkoxy, C1-C4 haloalkyl, C1-C4 haloalkoxy, cyano.

8. A compound or a pharmaceutically acceptable salt thereof according to any of claims 1-7 selected from a group consisting of:
2-oxo-3-phenyl-N-[4-(2-pyridinyl)-1,3-thiazol-2-yl]-1-oxa-3,8-diazaspiro[4.5]decane-8-carboxamide;
3-(3,4-dichlorophenyl)-2-oxo-N-[4-(2-pyridinyl)-1,3-thiazol-2-yl]-1-oxa-3,8-diazaspiro[4.5]decane-8-carboxamide;
(trans)-2-oxo-3-phenyl-N-[4-(2-pyridinyl)-1,3-thiazol-2-yl]-1-oxa-3-azaspiro[4.5]decane-8-carboxamide;
(trans)-2-oxo-3-(2-pyridinyl)-N-[4-(2-pyridinyl)-1,3-thiazol-2-yl]-1-oxa-3-azaspiro-[4.5]decane-8-carboxamide;
(trans)-N-[1-(2-fluorophenyl)-1H-pyrazol-3-yl]-2-oxo-3-phenyl-1-oxa-3-azaspiro[4.5]decane-8-carboxamide;
(trans)-N-[1-(2-fluorophenyl)-1H-pyrazol-3-yl]-2-oxo-3-(3-pyridinyl)-1-oxa-3-azaspiro[4.5]decane-8-carboxamide;
(trans)-N-[1-(2-fluorophenyl)-1H-pyrazol-3-yl]-2-oxo-3-(3-pyridazinyl)-1-oxa-3-azaspiro[4.5]decane-8-carboxamide;
(trans)-2-oxo-3-phenyl-N-(5-phenyl-2-pyrazinyl)-1-oxa-3-azaspiro[4.5]decane-8-carboxamide;
(trans)-N-(5-bromo-2-pyrazinyl)-2-oxo-3-phenyl-1-oxa-3-azaspiro[4.5]decane-8-carboxamide;
(trans)-2-oxo-3-phenyl-N-[5-(1,3-thiazol-2-yl)-2-pyrazinyl]-1-oxa-3-azaspiro[4.5]decane-8-carboxamide;
(trans)-N-[5-(2-fluorophenyl)-2-pyrazinyl]-2-oxo-3-phenyl-1-oxa-3-azaspiro[4.5]decane-8-carboxamide;
(trans)-2-oxo-3-phenyl-N-[5-(2-pyridinyl)-2-pyrazinyl]-1-oxa-3-azaspiro[4.5]decane-8-carboxamide;
(trans)-N-[5-(2-Fluorophenyl)-2-pyrimidinyl]-2-oxo-3-phenyl-1-oxa-3-azaspiro[4.5]decane-8-carboxamide;
(trans)-2-oxo-3-phenyl-N-3-quinolinyl-1-oxa-3-azaspiro[4.5]decane-8-carboxamide;
(trans)-2-oxo-3-(2-pyridinyl)-N-3-quinolinyl-1-oxa-3-azaspiro[4.5]decane-8-carboxamide;
(trans)-2-oxo-3-phenyl-N-2-quinoxalinyl-1-oxa-3-azaspiro[4.5]decane-8-carboxamide.

9. A method of treating a condition for which modulation of NPY Y5 receptors is beneficial, which comprises administering to a mammal (e.g. human) in need thereof an effective amount of a compound of any of claims 1-8.

10. A method as claimed in claim 9, wherein the condition is eating disorders.

11. A method as claimed in claim 10, wherein the condition is binge eating.

12. A method as claimed in claim 10, wherein the condition is obesity.

13. A method as claimed in claim 9, wherein the condition is depression.

14. Use of a compound as claimed in any of claims 1-8 in the manufacture of a medicament for the treatment of a condition in a mammal for which modulation of NPY-Y5 receptors is beneficial.

15. Use as claimed in claim 14, wherein the condition is eating disorders.

16. Use as claimed in claim 15, wherein the condition is binge eating.

17. Use as claimed in claim 15, wherein the condition is obesity.

18. Use as claimed in claim 14, wherein the condition is depression.

19. A compound as claimed in any of claims 1-8 for use in therapy.

20. A compound as claimed in any of claims 1-8 for use in the treatment of a condition in a mammal for which modulation of NPY-Y5 receptor is beneficial.

21. A compound as claimed in any of claims 1-8 for use in the treatment of an eating disorder.

22. A compound as claimed in any of claims 1-8 for use in the treatment of binge eating.

23. A compound as claimed in any of claims 1-8 for use in the treatment of binge eating.

24. A compound as claimed in any of claims 1-8 for use in the treatment of depression.

25. A pharmaceutical composition comprising a compound as claimed in any of claims 1-8 and a pharmaceutically acceptable carrier.
